(19) United States
(12) Patent Application Publication
(10) Pub. No.: US 2025/0180584 A1
(43) Pub. Date: Jun. 5, 2025

(54) ISOLATION AND DIAGNOSTIC METHODS USING CELL TYPE-SPECIFIC AND/OR ORGAN-SPECIFIC EXTRACELLULAR VESICLE (EV) MARKERS

(71) Applicants: President and Fellows of Harvard College, Cambridge, MA (US); The Brigham and Women's Hospital, Inc., Boston, MA (US)

(72) Inventors: Dmitry Ter-Ovanesyan, Cambridge, MA (US); Siddharth Iyer, Cambridge, MA (US); George M. Church, Cambridge, MA (US); David R. Walt, Boston, MA (US)

(73) Assignees: President and Fellows of Harvard College, Cambridge, MA (US); The Brigham and Women's Hospital, Inc., Boston, MA (US)

(21) Appl. No.: 18/937,363

(22) Filed: Nov. 5, 2024

Related U.S. Application Data
(63) Continuation of application No. PCT/US2023/022256, filed on May 15, 2023.
(60) Provisional application No. 63/342,353, filed on May 16, 2022.

(51) Int. Cl. G01N 33/68; C12Q 1/6883

(52) U.S. Cl. CPC G01N 33/6896; C12Q 1/6883; G01N 2800/50; G01N 2800/52

(57) ABSTRACT

The present invention relates to novel biomarkers and combinations thereof for cell type-specific and/or organ-specific extracellular vesicles, in particular, brain-specific and/or neuron-specific extracellular vesicles. The present invention also provides methods for isolation and/or enrichment of cell type-specific and/or organ-specific extracellular vesicles, methods for identification of extracellular vesicles derived from a cell, and methods for diagnosing or prognosing a disorder, e.g., a neurodegenerative disorder, using the cell type specific and/or organ-specific extracellular vesicles. Compositions in the form of kits of reagents for detecting the cell type-specific and/or organ-specific extracellular vesicles are also provided.

### RELATED APPLICATIONS

This application is a continuation of International Application No. PCT/US2023/022256, filed on May 15, 2023, which claims the benefit of priority to U.S. Provisional Application No. 63/342,353, filed on May 16, 2022. The entire contents of each of the foregoing applications are incorporated herein by reference.

### GOVERNMENT SUPPORT

This invention was made with government support under HG008525 awarded by National Institutes of Health (NIH). The government has certain rights in this invention.

### BACKGROUND OF THE INVENTION

Efforts to understand how the human brain functions are hampered partly due to the inability to perform brain biopsies on living individuals. Current understanding of brain diseases relies mainly upon postmortem tissue analysis after neurodegeneration and cell death have already occurred. Therefore, fundamental questions about the underlying biochemical processes of neurological diseases still remain. Having access to proteomic and transcriptomic profiles of neurons and other brain cells in living human individuals would be an asset to current understanding of neuroscience.

One approach to learning about the living brain is to analyze extracellular vesicles (EVs). EVs are released by many cell types and are found in all biological fluids. Since EVs contain RNA and protein from their donor cells, EVs represent a rich potential source of biomarkers. A grand challenge in maximizing the potential of EVs in molecular diagnostics is the isolation of cell-type specific EVs (Shah R, Patel T, et al., The New England Journal of Medicine. 2018; 379 (10): 958-66). First, EVs are heterogeneous and difficult to quantify. EVs and their contents are present at low amounts in clinically relevant biosamples where volumes are limited. Additionally, and partly due to a lack of suitable quantification methods, there is a lack of consensus about the best way of purifying EVs from plasma and other biofluids. Moreover, although the total population of EVs can be isolated from plasma or other biological fluids, the profiling of RNA and protein cargo of these EVs does not distinguish which cargo molecule comes from which cell type. Isolating EVs from a specific cell type would allow one to analyze the RNA and protein inside those EVs as a non-invasive “snapshot” of that cell type.

The ability to isolate EVs from neurons or other cell types of the brain would be particularly useful. Since the brain is inaccessible to biopsy, isolating neuron-derived EVs could allow for a readout of the state of the brain, as well as the development of biomarkers for early detection of neurodegenerative disease (Mustapic M, et al., Front Neurosci. 2017; 11:278; Hornung S, et al., Front Mol Neurosci. 2020; 13:38). Over the past several years, a number of studies have reported that the use of transmembrane protein LICAM, a cell adhesion molecule implicated in neural development, as a handle for EV capture. However, L1CAM may not be a suitable marker for neuron-derived EVs given its wide expression outside the brain on non-neuronal cells (Norman M. et al., Nature Methods. 2021, 18:631-634).

Thus, there is a need in the art for the identification of new biomarkers for cell type-specific EVs, in particular, brain-specific and/or neuron-specific EVs, which can be used to enable a better diagnosis or prognosis of diseases, such as neurodegenerative diseases, as well as for improved prediction of treatment outcomes.

### SUMMARY OF THE INVENTION

The present invention is based, at least in part, on the discovery of novel biomarkers for isolation of cell type-specific and/or organ-specific EV markers, e.g., brain-specific and/or neuron-specific EVs, from human biological samples, such as cerebrospinal fluid (CSF) or plasma. In particular, the invention is based on the surprising discovery that markers in any one of Tables 1-5 are specifically expressed in brain-specific and/or neuron-specific EVs and, thus, providing a path forward for the isolation of brain-specific and/or neuron-specific EVs from human samples. Furthermore, these novel biomarkers are useful for the identification of EVs derived from a specific organ and/or cell type, e.g., a brain cell, e.g., a neuron, an astrocyte, an oligodendrocyte, or a microglial cell, from a sample, e.g., a biological sample, for example, by determining the presence or absence of the markers in any one of Tables 1-5 on the surface of the EVs.

Accordingly, in one aspect, the present invention provides a method for isolating cell type-specific and/or organ-specific extracellular vesicles from a subject, comprising (a) obtaining a biological sample from the subject; and (b) isolating the cell type-specific and/or organ-specific extracellular vesicles based on the presence of a biomarker on the surface of the extracellular vesicles, wherein the biomarker comprises one or more biomarkers selected from Tables 1-5.

In some embodiments, the biological sample comprises a liquid biological sample.

In some embodiments, the liquid biological sample is selected from the group consisting of whole blood, serum, plasma, cerebrospinal fluid, spinal fluid, amniotic fluid, aqueous humor, vitreous humor, bile, breast milk, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof.

In some embodiments, the extracellular vesicles are brain-specific. In some embodiments, the extracellular vesicles are neuron-specific, astrocyte-specific, oligodendrocyte-specific, and/or microglial-specific.

In some embodiments, the extracellular vesicles are neuron-specific and wherein the one or more biomarkers are selected from Tables 1 and 5.

In some embodiments, the one or more biomarkers are selected from the group consisting of GABRG2, SVOP, SLC32A1, GRM7, GABRB3, CHRNB2, SLC12A5, GRM8, PTPRT, SLC6A17, PCDHAC2, PLPPR4, C11orf87, SORCS3, CALY, PTPRR, KIAA1549L, HCN1, CDH18, TMEM132D, GPR158, FRRS1L, ATP2B3, GRIA4, ST8SIA3, HS6ST3, SEZ6, and NRXN3.

In some embodiments, the extracellular vesicles are astrocyte-specific and wherein the one or more biomarkers are selected from Table 2.

In some embodiments, the extracellular vesicles are oligodendrocyte-specific and wherein the one or more biomarkers are selected from Table 3.

In some embodiments, the extracellular vesicles are microglial-specific and wherein the one or more biomarkers are selected from Table 4.

In some embodiments, the cell type-specific and/or organ-specific EVs are isolated by immuno-isolation, mixed-mode chromatography, size exclusion chromatography, cation exchange chromatography, anion exchange chromatography, gel permeation chromatography, differential centrifugation, sucrose density gradients, organelle electrophoresis, magnetic activated cell sorting (MACS), or nanomembrane ultrafiltration concentrator.

In some embodiments, the immuno-isolation comprises a microfluidic affinity-based isolation, a magnetic based isolation, a pull-down isolation, or a fluorescence activated sorting-based isolation.

In one aspect, the present invention provides a method for isolating brain-specific extracellular vesicles from a subject, comprising (a) obtaining a biological sample from the subject; (b) isolating extracellular vesicles from the sample based on the presence of a biomarker on the surface of the extracellular vesicles, wherein the biomarker comprises one or more biomarkers selected from Tables 1-5.

In some embodiments, the biological sample comprises a liquid biological sample.

In some embodiments, the liquid biological sample is selected from the group consisting of whole blood, serum, plasma, cerebrospinal fluid, spinal fluid, amniotic fluid, aqueous humor, vitreous humor, bile, breast milk, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof.

In some embodiments, the extracellular vesicles are neuron-specific, astrocyte-specific, oligodendrocyte-specific, and/or microglial-specific.

In some embodiments, the extracellular vesicles are neuron-specific and wherein the one or more biomarkers are selected from Tables 1 and 5.

In some embodiments, the one or more biomarkers are selected from the group consisting of GABRG2, SVOP, SLC32A1, GRM7, GABRB3, CHRNB2, SLC12A5, GRM8, PTPRT, SLC6A17, PCDHAC2, PLPPR4, C11orf87, SORCS3, CALY, PTPRR, KIAA1549L, HCN1, CDH18, TMEM132D, GPR158, FRRS1L, ATP2B3, GRIA4, ST8SIA3, HS6ST3, SEZ6, and NRXN3.

In some embodiments, the extracellular vesicles are astrocyte-specific and wherein the one or more biomarkers are selected from Table 2.

In some embodiments, the extracellular vesicles are oligodendrocyte-specific and wherein the one or more biomarkers are selected from Table 3.

In some embodiments, the extracellular vesicles are microglial-specific and wherein the one or more biomarkers are selected from Table 4.

In some embodiments, the brain-specific EVs are isolated by immuno-isolation, mixed-mode chromatography, size exclusion chromatography, cation exchange chromatography, anion exchange chromatography, gel permeation chromatography, differential centrifugation, sucrose density gradients, organelle electrophoresis, magnetic activated cell sorting (MACS), or nanomembrane ultrafiltration concentrator.

In some embodiments, the immuno-isolation comprises a microfluidic affinity based isolation, a magnetic based isolation, a pull-down isolation, or a fluorescence activated sorting-based isolation.

In one aspect, the present invention provides a method for identifying an extracellular vesicle derived from a brain cell, comprising (a) obtaining a biological sample comprising the extracellular vesicle; (b) determining the presence or absence of a biomarker on the surface of the extracellular vesicle, wherein the biomarker comprises one or more biomarkers selected from Tables 1-5; and wherein the presence of the biomarker is indicative of the extracellular vesicle being derived from a brain cell.

In some embodiments, the brain cell is selected from a group consisting of a neuron, an astrocyte, an oligodendrocyte, and a microglial cell.

In one aspect, the present invention provides a method for identifying an extracellular vesicle derived from a neuron, comprising (a) obtaining a biological sample comprising the extracellular vesicle; (b) determining the presence or absence of a biomarker on the surface of the extracellular vesicle, wherein the biomarker comprises one or more biomarkers selected from Tables 1 and 5; and wherein the presence of the biomarker is indicative of the extracellular vesicle being derived from a neuron.

In some embodiments, the one or more biomarkers are selected from the group consisting of GABRG2, SVOP, SLC32A1, GRM7, GABRB3, CHRNB2, SLC12A5, GRM8, PTPRT, SLC6A17, PCDHAC2, PLPPR4, C11orf87, SORCS3, CALY, PTPRR, KIAA1549L, HCN1, CDH18, TMEM132D, GPR158, FRRS1L, ATP2B3, GRIA4, ST8SIA3, HS6ST3, SEZ6, and NRXN3.

In another aspect, the present invention provides a method for identifying an extracellular vesicle derived from an astrocyte, comprising (a) obtaining a biological sample comprising the extracellular vesicle; (b) determining the presence or absence of a biomarker on the surface of the extracellular vesicle, wherein the biomarker comprises one or more biomarkers selected from Table 2; and wherein the presence of the biomarker is indicative of the extracellular vesicle being derived from an astrocyte.

In one aspect, the present invention provides a method for identifying an extracellular vesicle derived from an oligodendrocyte, comprising (a) obtaining a biological sample comprising the extracellular vesicle; (b) determining the presence or absence of a biomarker on the surface of the extracellular vesicle, wherein the biomarker comprises one or more biomarkers selected from Table 3; and wherein the presence of the biomarker is indicative of the extracellular vesicle being derived from an oligodendrocyte.

In another aspect, the present invention provides a method for identifying an extracellular vesicle derived from a microglial cell, comprising (a) obtaining a biological sample comprising the extracellular vesicle; (b) determining the presence or absence of a biomarker on the surface of the extracellular vesicle, wherein the biomarker comprises one or more biomarkers selected from Table 4; and wherein the presence of the biomarker is indicative of the extracellular vesicle being derived from a microglial cell.

In some embodiments, the biological sample comprises a liquid biological sample.

In some embodiments, the liquid biological sample is selected from the group consisting of whole blood, serum, plasma, cerebrospinal fluid, spinal fluid, amniotic fluid, aqueous humor, vitreous humor, bile, breast milk, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof.

In some embodiments, the biological sample is obtained from a subject.

In some embodiments, the presence or absence of the biomarker is determined by RNA sequencing (RNA seq), DNA sequencing, array analysis, reverse transcription polymerase chain reaction (RT-PCR), quantitative reverse transcription polymerase chain reaction (qRT-PCR), proteomic profiling, mass spectrometry, immunoassay, ELISA, fluorescence activated cell sorting (FACS), SDS-polyacrylamide gel electrophoresis (SDS-PAGE), or Western blot analysis.

In one aspect, the present invention provides a method for diagnosing, prognosing, or identifying a subject at risk of developing a neurodegenerative disorder in a subject comprising: (a) obtaining a biological sample from the subject; (b) isolating brain-specific extracellular vesicles from the biological sample based on the presence of a biomarker in the isolated extracellular vesicles, wherein the biomarker comprises one or more biomarkers selected from Tables 1-5; (c) extracting protein and/or nucleic acids from the isolated brain-specific extracellular vesicles; and (d) analyzing the extracted protein and/or nucleic acids from the isolated brain-specific extracellular vesicles, thereby diagnosing, prognosing, or identifying the subject at risk of developing the neurodegenerative disorder.

In some embodiments, the biological sample comprises a liquid biological sample.

In some embodiments, the liquid biological sample is selected from the group consisting of whole blood, serum, plasma, cerebrospinal fluid, spinal fluid, amniotic fluid, aqueous humor, vitreous humor, bile, breast milk, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof.

In some embodiments, the extracellular vesicles are neuron-specific, astrocyte-specific, oligodendrocyte-specific, and/or microglial-specific.

In some embodiments, the extracellular vesicles are neuron-specific and wherein the one or more biomarkers are selected from Tables 1 and 5.

In some embodiments, the one or more biomarkers are selected from the group consisting of GABRG2, SVOP, SLC32A1, GRM7, GABRB3, CHRNB2, SLC12A5, GRM8, PTPRT, SLC6A17, PCDHAC2, PLPPR4, C11orf87, SORCS3, CALY, PTPRR, KIAA1549L, HCN1, CDH18, TMEM132D, GPR158, FRRS1L, ATP2B3, GRIA4, ST8SIA3, HS6ST3, SEZ6, and NRXN3.

In some embodiments, the extracellular vesicles are astrocyte-specific and wherein the one or more biomarkers are selected from Table 2.

In some embodiments, the extracellular vesicles are oligodendrocyte-specific and wherein the one or more biomarkers are selected from Table 3.

In some embodiments, the extracellular vesicles are microglial-specific and wherein the one or more biomarkers are selected from Table 4.

In some embodiments, the extracted nucleic acids comprise messenger RNA (mRNA), microRNA (miRNA), long non-coding RNA (lncRNA), small non-coding RNA, DNA, and any other full length or fragment of RNAs or DNAs.

In some embodiments, analyzing the extracted nucleic acids from the isolated brain-specific extracellular vesicles comprises RNA sequencing (RNA seq), DNA sequencing, array analysis, reverse transcription polymerase chain reaction (RT-PCR), or quantitative reverse transcription polymerase chain reaction (qRT-PCR).

In some embodiments, analyzing the extracted nucleic acids from the isolated brain-specific extracellular vesicles comprises genome-wide analysis, or transcriptome profiling.

In some embodiments, analyzing the extracted nucleic acids from the isolated brain-specific extracellular vesicles comprises analyzing a gene of interest, wherein the gene of interest is associated with the neurodegenerative disorder.

In some embodiments, the methods comprise testing for the presence or absence of said gene of interest, analyzing for one or more allelic variants or mutations of the gene of interest, testing for presence or absence of the allelic variants or mutations.

In some embodiments, analyzing the extracted protein from the isolated brain-specific extracellular vesicles comprises proteomic profiling, mass spectrometry, immunoassay, ELISA, fluorescence activated cell sorting (FACS), SDS-polyacrylamide gel electrophoresis (SDS-PAGE), or Western blot analysis.

In some embodiments, analyzing the extracted protein from the isolated brain-specific extracellular vesicles comprises analyzing a protein of interest, wherein the protein of interest is associated with the neurodegenerative disorder.

In some embodiments, the methods comprise testing for the presence or absence of said protein of interest, analyzing for one or more mutations in the protein of interest, testing for presence or absence of the mutations.

In some embodiments, the neurodegenerative disorder is selected from the group consisting of: Alzheimer's disease (AD), Huntington's Disease, multiple sclerosisvascular disease dementia, frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease.

In one aspect, the present invention provides a kit for isolating brain-specific extracellular vesicles from a subject, comprising (a) one or more reagents for detecting the presence of a biomarker on the surface of the extracellular vesicle, wherein the biomarker comprises one or more biomarkers selected from Tables 1-5; (b) means for isolating the brain-specific extracellular vesicles based on the presence of the biomarker; and (c) a set of instructions for detecting the presence of the biomarker, and/or isolating the brain-specific extracellular vesicles.

In some embodiments, the one or more reagents for detecting the presence of the biomarker on the extracellular vesicles is an antibody or an aptamer that binds the biomarker.

In some embodiments, the kits further comprise means for isolating a biological sample from the subject.

In another aspect, the present invention provides a kit for detecting a neurodegenerative disorder in a subject, comprising (a) one or more reagents for detecting the presence of a biomarker on the surface of the extracellular vesicle, wherein the biomarker comprises one or more biomarkers selected from Tables 1-5; (b) means for isolating the brain-specific extracellular vesicles based on the presence of the biomarker; (c) one or more reagents for detecting the level of a gene associated with the neurodegenerative disorder in the isolated brain-specific extracellular vesicles; and (d) a set of instructions for detecting the presence of the biomarker, isolating the brain-specific extracellular vesicles, and/or detecting the level of the gene associated with the neurodegenerative disorder.

In some embodiments, the kits further comprise means for isolating a biological sample from the subject.

Other features and advantages of the invention will be apparent from the following detailed description and figures.

### BRIEF DESCRIPTION OF THE DRAWINGS

FIGS. 1A-1E depict the identification of neuron specific transmembrane proteins and the characterization of extracellular vesicles from human iPS-derived neurons. FIG. 1A is a schematic depicting the immuno-isolation of neuron-derived EVs from human CSF or plasma. FIG. 1B is a scatter plot of Tau scores for genes where that are maximally expressed in neurons relative to other cell types of the brain and the brain relative to other organs. L1CAM is indicated relative to other genes. FIG. 1C depicts the overlap of transmembrane proteins expressed specifically in neurons relative to other cell types of the brain (using a Tau specificity score of 0.7 or above) and in the brain relative to other organs in the body (also using a Tau specificity score of 0.7 or above). FIG. 1D depicts the isolation of EVs from conditioned media of undifferentiated human iPS cells or iPS-derived neurons and profiling of their proteins using mass spectrometry to determine the presence of transmembrane proteins (2 peptides or more) on neuron EVs but not iPS EVs. FIG. 1E depicts the overlap of brain-specific transmembrane proteins, neuron-specific transmembrane proteins, and transmembrane proteins detected in neuron EVs by mass spectrometry. FIG. 1F depicts the validation by Western blotting of candidate marker SYT1 in iNGN neurons and iNGN neuron EVs.

FIGS. 2A-2H depict immuno-isolation of EVs from cell culture and human biofluids FIG. 2A is a schematic depicting immuno-isolation of EVs from the cell culture media of K562 cells. FIG. 2B is the Western blotting image of CD81 after immuno-isolation of EVs. Equal amounts of EVs obtained by differential ultracentrifugation of K562 cell culture media were loaded as input or immuno-isolated using either an anti-CD81 antibody or a control non-specific antibody. PD=pulldown, FT=flow-through. FIG. 2C is the Western blotting image of L1CAM after immuno-isolation of EVs. Equal amounts of EVs obtained by differential ultracentrifugation of iNGN neuron cell culture media were loaded as input or immuno-isolated using either an anti-L1CAM antibody or a control non-specific antibody. PD=pulldown, FT=flow-through. FIG. 2D is a schematic depicting the EV mixing experiment using EVs isolated from the conditioned media of iPS cells and iPS-derived iNGN neurons. FIG. 2E is a Western blotting image of L1CAM and GJA1 after immuno-isolation using anti-L1CAM antibodies from pooled iNGN neuron EVs and iPS EVs mixed in varying ratios (normalized relative to cell number). PD=pulldown, FT=flow-through. FIG. 2F is a schematic depicting immuno-isolation of EVs from human plasma or CSF. EVs were immuno-isolated from biofluid without initial EV purification. Immuno-isolation was performed using a target antibody or control antibody (PD1=pulldown 1) and the flow-through was subjected to a second immuno-isolation with the target antibody (PD2=pulldown 2). FIG. 2G shows Western blots of CD9 (bottom), CD63 (middle), or CD81 (top) after immuno-isolation from human CSF. In each case, the first immuno-isolation (PD1) was performed with antibodies against the target protein or a control antibody, and the second immuno-isolation (PD2) was performed with antibodies against the target protein using the flow-through of PD1. FIG. 2H shows Western blots of CD9 (bottom), CD63 (middle), or CD81 (top) after immuno-isolation from human plasma. In each case, the first immuno-isolation (PD1) was performed with antibodies against the target protein or a control antibody, and the second immuno-isolation (PD2) was performed with antibodies against the target protein using the flow-through of PD1.

FIGS. 3A-3G depict the purification of EVs from human biofluids using mixed-mode resin (MMR) slurry. FIG. 3A is a schematic illustrating MMR slurry technique. Capto Core 700 beads have pores allowing biomolecules less than 700 kDa to enter, and once free proteins enter, they stay in the beads, which can be removed to leave pure EVs. FIG. 3B is a schematic depicting EV isolation workflow with MMR slurry. FIG. 3C are Western blots of CD9, CD63 and CD81 and albumin in human cerebrospinal fluid after MMR slurry purification with increasing amounts of Capto Core beads. FIG. 3D is Western blot of L1CAM in CSF after MMR slurry purification with increasing amounts of Capto Core beads. FIG. 3E depicts the total protein stain of CSF after MMR slurry purification with increasing amounts of Capto Core beads. FIG. 3F depicts the Western blots of CD9, CD63 and CD81 and albumin after size exclusion chromatography of plasma, followed by MMR slurry purification with increasing amounts of Capto Core beads. FIG. 3 depicts the total protein stain after size exclusion chromatography of plasma, followed by MMR slurry purification with increasing amounts of Capto Core beads. Ratio of Capto Core (CC) beads to proteins is indicated in μL CC slurry/μg protein in sample.

FIGS. 4A-4E depict that establishment of the EV proteome in human biofluids yields novel neuron-specific EV markers. FIG. 4A is a schematic depicting EV proteomics using MMR slurry for CSF and SEC followed by MMR slurry in plasma. FIG. 4B depicts the number of distinct proteins total and transmembrane proteins detected in human (pooled) CSF and plasma. FIG. 4C depicts the general pipeline for identification of neurons-specific EV markers: assessing transmembrane proteins for neuron-specific gene expression and presence on EVs (in EV proteomics data). FIG. 4D depicts that using a Tau cutoff of 0.7 for neuron and brain-specific expression together with proteins found on EVs in MMR slurry proteomics data yields 27 candidate neurons-specific EV markers. FIG. 4E depicts a list of 27 candidate neurons-specific EV markers with their Tau values for specificity of expression in neurons relative to other cell types of the brain and the brain relative to other organs in the body, as well as presence in CSF and/or plasma mass spectrometry data.

FIG. 5 depicts a heatmap of Tau cutoffs. Number of transmembrane proteins when different cutoffs are used of Tau for neurons within the brain and the brain relative to other cells of the body

FIG. 6 depicts a scatterplot of Tau for all genes. Tau specificity scores for all genes for neurons (in Brain RNA-Seq dataset) and brain (in GTEx).

FIG. 7 depicts a scatterplot of Tau for genes with maximum expression in brain and neurons. Tau specificity scores for genes with maximum expression in both neurons relative to other cells of the brain (in Brain RNA-Seq dataset) and brain relative to other organs (in GTEx).

FIGS. 8A-E depict the microscopic images of iPS iNGN cell differentiation into neurons. FIG. 8A depicts iPS NGN cells before doxycycline addition (40× magnification). FIG. 8B depicts iPS NGN cells 1 day after doxycycline addition (40× magnification). FIG. 8C depicts iPS NGN cells 2 days after doxycycline addition (40× magnification). FIG. 8D depicts iPS NGN cells 4 days after doxycycline addition (40× magnification). FIG. 8E depicts iPS NGN cells 5 days after doxycycline addition (200× magnification).

FIG. 9 depicts that direct immuno-isolation of EVs works better than indirect isolation for CD81. Direct (primary antibody conjugated to beads and then added to sample) vs. indirect (primary antibody is added to sample first and then added to beads) pulldown methods are compared for CD81 immuno-isolation using two different antibodies (clones M38 and 1.3.3.22). Note: band at 150 kDa is primary antibody coming off of beads.

FIG. 10 depicts the effect of immuno-isolation incubation time and temperature on CD81 capture efficiency. Western blot against CD81 and CD63 after EVs were incubated with beads for either 1 hour or overnight (ON), at different temperatures (4C, room temperature, or 37C) using beads conjugated to either CD81 or a control (ctrl) non-specific antibody (against mCherry).

FIG. 11 depicts that optimized immuno-isolation protocol is efficient and specific against K562 EVs with CD81 or CD63. Western blot against CD81 and CD63 after EVs were incubated with beads for 1 hour at 37C using beads conjugated to antibody against CD81, CD63 or mCherry. As an extra control to ensure that all EVs came off of beads during lysis, beads were reboiled after lysis to see if more material was left. Additionally, EV pellet was treated with Proteinase K (PK) or Triton X and Proteinase K.

FIG. 12 depicts the optimal immuno-isolation conditions for CD81 not optimal for L1CAM. Immuno-isolation for 1 hour at 37C was performed on K562 EVs using CD81 (or control GFP antibody) and neuron EVs using L1CAM (or control GFP antibody) and corresponding western blot was performed against CD81 (Top) or L1CAM (Bottom). Intensity of bands on western blot was quantified as percentage of band relative to sum of pulldown (PD)+flow-through (FT) for that condition.

FIG. 13 depicts the effect of immuno-isolation incubation time, volume and temperature on L1CAM capture efficiency. Western blot against L1CAM after neuron EVs were incubated with antibody conjugated beads (against L1CAM or GFP) for different times (1, 2, 4, or 24 hours), in different volumes (0.5 mL or 2 mL) at different temperatures (4C or 37C). Intensity of bands on western blot was quantified as percentage of band relative to sum of pulldown (PD)+flow-through (FT) for that condition.

FIGS. 14A-B depict the comparison of different immuno-isolation beads and conditions for human biofluids. FIG. 14A depicts that Goat anti-mouse (GAM) dynabeads were compared to Sheep anti-mouse (SAM) dynabeads in terms of pulldown of CD81 in plasma. Pulldown incubation temperature and GAM dynabead amount were also compared. FIG. 14B depicts that Goat anti-mouse (GAM) dynabead pulldown incubation temperature and incubation with or without BSA were also compared in terms of pulldown of CD81 in CSF.

FIGS. 15A-E depict that Capto Core Slurry Purification method conditions are optimized for maximum exosome recovery to albumin depletion and is superior to other isolation methods. FIG. 15A depicts that either 100 μL (Low CC) or 300 μL (High CC) 50% Capto Core slurry was added to K562 exosomes, and incubated either once (1×), or two times in succession end over end for 45 minutes at room temperature. FIG. 15B depicts that CSF was incubated with 50% Capto Core slurry in a time series to optimize incubation time. FIG. 15C depicts that different amounts of 50% capto core slurry and amounts of CSF were tested in combinations to ascertain whether binding capacity or volume/volume ratio is more important. CC1:0.5 mL CSF-160 uL CC; CC2:1 mL CSF-160 uL CC; CC3:0.1 mL CSF/0.9 mL PBS-160 uL CC; CC4:0.5 mL CSF/0.5 mL PBS-160 uL CC; CC5:0.1 mL CSF-16 uL CC; CC6:0.5 mL CSF-80 uL CC; CC7:0.1 mL CSF/0.9 mL PBS-16 uL CC; CC8:0.5 mL CSF/0.5 mL PBS-80 uL CC; CC9:0.1 mL CSF/0.9 mL PBS-40 uL CC; CC10:0.5 mL CSF/0.5 mL PBS-40 uL CC; CC11:1 mL CSF-40 uL CC; CC12:0.1 mL CSF/0.9 mL PBS-320 uL CC; CC13:0.5 mL CSF/0.5 mL PBS-320 uL CC; CC14:1 mL CSF-320 uL CC. FIG. 15D depicts that Capto Core tetraspanin yield is compared to size exclusion chromatography (Izon and Sepharose 4B and 6B columns) tetraspanin yield. FIG. 15E depicts that Capto Core albumin depletion is compared to size exclusion chromatography albumin depletion by Acqua commassie blue stain, and Capto Core albumin depletion is assessed by Ponceau S.

FIG. 16A depicts the detection of NRXN3 recombinant protein standard by the Simoa assay. Invitrogen PA5-71367 was used as the capture antibody and CST 480045 was used as the detector antibody. Average enzyme per bead (AEB) is plotted as a function of concentration of NRXN3 recombinant protein standard. FIG. 16B depicts the detection of NRXN3 in early size exclusion chromatography (SEC) fractions by the Simoa NRXN3 assay from EVs ultracentrifuged from conditioned media of human iPS-derived neurons. FIG. 16C depicts the detection of NRXN3 in early size exclusion chromatography (SEC) fractions by the Simoa NRXN3 assay in human CSF samples.

### DETAILED DESCRIPTION OF THE INVENTION

The present invention is based, at least in part, on the discovery of novel biomarkers for isolation of cell type-specific and/or organ-specific EV markers, e.g., brain-specific and/or neuron-specific EVs, from human biological samples, such as cerebrospinal fluid (CSF) or plasma. In particular, the invention is based on the surprising discovery that markers in any one of Tables 1-5 are specifically expressed in brain-specific and/or neuron-specific EVs, thus providing a path forward for the isolation of brain-specific and/or neuron-specific EVs from human samples. Furthermore, these novel biomarkers are useful for the identification of EVs derived from a specific cell type, e.g., a brain cell, e.g., a neuron, an astrocyte, an oligodendrocyte, or a microglial cell, from a sample, e.g., a biological sample, for example, by determining the presence or absence of the markers in any one of Tables 1-5 on the surface of the EVs.

Various aspects of the invention are described in further details in the following subsections:

### A. Definition

In order that the present disclosure may be more readily understood, certain terms are first defined. It should also be noted that whenever a value or range of values of a parameter are recited, it is intended that values and ranges intermediate to the recited values are also part of this disclosure.

In the following description, for purposes of explanation, specific numbers, materials, and configurations are set forth in order to provide a thorough understanding of the disclosure. It will be apparent, however, to one having ordinary skill in the art that the disclosure may be practiced without these specific details. In some instances, well-known features may be omitted or simplified so as not to obscure the present disclosure. Furthermore, reference in the specification to phrases such as “one embodiment” or “an embodiment” mean that a particular feature, structure, or characteristic described in connection with the embodiment is included in at least one embodiment of the disclosure. The appearances of phrases such as “in one embodiment” in various places in the specification are not necessarily all referring to the same embodiment.

The articles “a” and “an” are used herein to refer to one or to more than one (i.e., to at least one) of the grammatical object of the article. By way of example, “an element” refers to one element or more than one element.

The term “comprising” or “comprises” is used herein in reference to compositions, methods, and respective component(s) thereof, that are essential to the disclosure, yet open to the inclusion of unspecified elements, whether essential or not.

As used herein, the term “one or more” or “at least one of” is understood as each value 1, 2, 3, 4, 5, 6, 7, 8, 9, 10, 11, 12, 13, 14, 15, 16, 17, 18, 19, and 20 and any value greater than 20.

As used herein, the term “biomarker” is, in one embodiment, a biological molecule, or a panel of biological molecules, or any combination thereof, which has an altered level in a test sample, e.g., a cell type-specific or an organ-specific EV, e.g., a brain- and/or neuron-specific EV, as compared to its level in a control sample, e.g., an EV from a different cell type or a different organ. Examples of biomarkers include, for example, polypeptides, peptides, polypeptide fragments, proteins, antibodies, hormones, polynucleotides, DNA, RNA or RNA fragments, microRNA (miRNAs), lipids, metabolites, or polysaccharides. In one embodiment, the biomarker is detected in a cell type-specific EV isolated from a biological sample. In one embodiment, the biomarker is an organ-specific EV marker, e.g., a marker from anus, arteries, appendix, adrenal glands, brain, bones, bronchi, bladder, bone marrow, bulbourethral glands, colon, cervix, clitoris, capillaries, cerebellum, diaphragm, ears, eyes, fallopian tubes, genitals, gallbladder, heart, hair follicle, hypothalamus, interstitium, kidneys, joints, liver, lungs, larynx, ligaments, lymph nodes, large intestine, lymphatic vessel, mouth, mesentery, mammary glands, nose, nails, nerves, nasal cavity, ovaries, esophagus, penis, pancreas, pharynx, placenta, prostate, pineal gland, pituitary gland, parathyroid glands, rectum, skin, spleen, scrotum, stomach, spinal cord, small intestine, salivary glands, skeletal muscles, seminal vesicles, subcutaneous tissue, teeth, tonsils, testes, tendons, tongue, thyroid, trachea, thymus gland, ureters, urethra, uterus, vulva, veins, vagina, vas deferens, or vestigial organ. In one embodiment, the biomarker is detected in a brain-specific EV. In one embodiment, the biomarker is detected in a neuron-specific EV. In one embodiment, the biomarker is detected in an astrocyte-specific EV. In one embodiment, the biomarker is detected in an oligodendrocyte-specific EV. In one embodiment, the biomarker is detected in a microglial-specific EV. In some embodiments, the biomarker comprises one or more markers selected from Tables 1-5.

A biomarker may be differentially present at any level, but is generally present at a level that is increased or decreased in a test sample, e.g., a cell type-specific or an organ-specific EV, e.g., a brain- and/or neuron-specific EV, isolated from a biological sample obtained from a subject, as compared to its level in a control sample, e.g., an EV from a different cell type or a different organ, by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more. A biomarker is preferably differentially present at a level that is statistically significant (e.g., a p-value less than 0.05 as determined using any statistical test). As such, the difference between the level of a biomarker of the present invention and a corresponding control or reference value can be a statistically significant value.

As used herein, “biological sample” refers to any biological sample obtained from or derived from a subject. In some embodiments, the biological sample comprises EVs. In another embodiment, the biological sample is a liquid biological sample. The term “liquid biological sample,” as used herein, refers to a sample that is substantially in liquid form. In some embodiments, a liquid sample is a body fluid. Body fluids include, e.g., whole blood (including fresh or frozen), peripheral blood, plasma (including fresh or frozen), serum (including fresh or frozen), cerebrospinal fluid (CSF), ascites, sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids. A biological sample may also include the blastocyst cavity, umbilical cord blood, or maternal circulation that may be of fetal or maternal origin. The biological sample may also be a tissue sample or biopsy, from which EVs may be obtained. In one embodiment, the biological sample is a brain tissue. In one embodiment, the biological sample is a plasma sample. In another embodiment, the biological sample is a CSF sample.

As used herein, “subject” refers to any animal. In some embodiments, the subject is a human. Other animals that can be subjects include but are not limited to non-human primates (e.g., monkeys, gorillas, and chimpanzees), domesticated animals (e.g., horses, pigs, donkeys, goats, rabbits, sheep, cattle, yaks, alpacas, and llamas), and companion animals (e.g., cats, dogs, hamsters, guinea pigs, rats, mice, and birds.)

### B. Extracellular Vesicles

The present invention provides biomarkers for cell type-specific and/or or organ-specific extracellular vesicles (EVs), e.g., brain- and/or neuron-specific EVs, methods for purification of cell type-specific and/or organ-specific EVs, and diagnostic and prognostic methods for diseases, such as neurodegenerative disorders, using these cell type-specific or organ-specific EVs.

Extracellular vesicles (EVs) are a class of membrane bound organelles secreted by various cell types. As used herein, the term “extracellular vesicle” refers to a cell-derived vesicle having a membrane that surrounds and encloses a central internal space. Membranes of EVs can be composed of a lipid bi-layer having an external surface and an internal surface bounding an enclosed volume. EVs are able to carry various molecules, such as proteins, lipids and RNAs on their surface as well as within their lumen.

EVs include all membrane-bound vesicles that have a cross-sectional diameter smaller than the cell from which they are secreted. In some embodiments, EVs can have a longest dimension, such as a longest cross-sectional dimension, such as a cross-sectional diameter ranging from 1 nm to 1000 nm, such as 10 nm to 1000 nm, such as 20 nm to 1000 nm, such as 30 nm to 1000 nm, such as 1 to 100 nm, such as 10 to 100 nm, such as 20 to 100 nm, such as 30 to 100 nm, such as 40 to 100 nm, such as 10 to 200 nm, such as 20 to 200 nm, such as 30 to 200 nm, such as 40 to 200 nm, such as 10 to 120 nm, such as 20 to 120 nm, such as 30 to 120 nm, such as 40 to 120 nm, such as 10 to 300 nm, such as 20 to 300 nm, such as 30 to 300 nm, such as 40 to 300 nm, such as 50 to 1000 nm, such as 500 to 2000 nm, such as 100 to 500 nm, such as 500 to 1000 nm and such as 40 nm to 500 nm, each range inclusive.

According to their size and density, extracellular vesicles can be divided into three main groups: exosomes (10-150 nm), microvesicles (100-1000 nm), and apoptotic bodies (1-10 μm). As used herein, the term “exosome” refers to a cell-derived vesicle composed of a membrane enclosing an internal space, wherein the vesicle is generated from a cell by fusion of the late endosome with the plasma membrane or by direct plasma membrane budding. An exosome is typically created intracellularly when a segment of the cell membrane spontaneously invaginates and is ultimately exocytosed. As used herein, exosomes can also include any shed membrane bound particle that is derived from either the plasma membrane or an internal membrane. Exosomes can also include cell-derived structures bounded by a lipid bilayer membrane arising from both blebbing and sealing of portions of the plasma membrane or from the export of any intracellular membrane-bounded vesicular structure containing various membrane-associated proteins, including surface-bound molecules derived from the host circulation that bind selectively to the exosomal proteins together with molecules contained in the exosome lumen, including but not limited to mRNAs, microRNAs or intracellular proteins. Blebs and blebbing are further described in Charras et al, Nature Reviews Molecular and Cell Biology, Vol. 9, No. 11, p. 730-736 (2008). Exosomes can also include membrane fragments.

EVs contain RNA, e.g., microRNAs (miRNA), long non-coding RNAs (lncRNA), mRNAs, DNA fragments, and proteins from their donor cells and, thus, are important for intercellular communications within the human body and involved in many pathophysiological conditions, such as neurodegenerative diseases. EVs can also be loaded with various drugs and exogenous nucleic acids or proteins and deliver this cargo to different cells. Importantly, EVs are natural carriers for miRNAs and other non-coding RNAs, and the direct membrane fusion with the target cell allows contents to be delivered directly into the cytosol. This makes EVs an excellent delivery system for small molecules.

EVs are abundant in various biological samples. In some embodiments, the sample is a sample obtained from a cell culture. In some embodiments, the sample is a liquid sample, e.g., a liquid biological sample. Exemplary liquid samples include, but are not limited to, body fluids, such as whole blood (including fresh or frozen), peripheral blood, plasma (including fresh or frozen), serum (including fresh or frozen), cerebrospinal fluid (CSF), ascites, sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids. A biological sample may also include the blastocyst cavity, umbilical cord blood, or maternal circulation that may be of fetal or maternal origin. The biological sample may also be a tissue sample or biopsy, from which EVs may be obtained. In one embodiment, the biological sample is a brain tissue. In one embodiment, the biological sample is a plasma sample. In another embodiment, the biological sample is a CSF sample. Methods or obtaining tissue biopsies and body fluids from mammals are well known in the art.

According to methods of the present invention, detection of EVs in various patient biological fluids allows for assessment of disease progression, immune response, and toxicity, and, thus, the isolation and detection of EVs can aid in disease diagnosis, prognosis, and monitoring of treatment response. A grand challenge in maximizing the potential of EVs in molecular diagnostics is the isolation of cell-type specific EVs (Shah R, Patel T, et al., The New England Journal of Medicine. 2018; 379 (10): 958-66). Although the total population of EVs can be isolated from plasma or other biological fluids, the profiling of RNA and protein cargo of these EVs does not distinguish which cargo molecule comes from which cell type. Isolating EVs from a specific cell type would allow one to analyze the RNA and protein inside those EVs as a non-invasive “snapshot” of that cell type.

The inventors of the present application, however, were able to discover novel biomarkers for isolation of cell-type and organ-specific EVs. In some embodiments, the EVs of the present invention are from a particular cell type, for example a brain cell type, e.g., a neuron, an astrocyte, an oligodendrocyte, or a microglial cell. In some embodiments, the EVs comprise neuron-derived EVs. In some embodiments, the EVs comprise astrocyte-derived EVs, oligodendrocyte-derived EVs, or microglial-derived EVs.

### C. Biomarkers of the Invention

The present invention is based, at least in part, on the discovery of novel biomarkers for isolation of cell type-specific and/or organ-specific EV markers, e.g., brain-specific and/or neuron-specific EVs, from human biological samples, such as cerebrospinal fluid (CSF) or plasma. In particular, the invention is based on the surprising discovery that markers in any one of Tables 1-5 are expressed in brain-specific and/or neuron-specific EVs. Thus, these differentially expressed markers are useful in isolating brain-specific and/or neuron-specific EVs from human samples.

Isolating EVs from a specific cell type allow one to analyze the RNA and protein inside those EVs as a non-invasive “snapshot” of that cell type. In particular, given the inaccessibility of brain for biopsy, the ability to isolate EVs from neurons or other cell types of the brain is particularly useful. For example, isolating neuron-derived EVs allows for a readout of the state of the brain, as well as the development of biomarkers for early detection of neurodegenerative disease. Thus, identification of new biomarkers for cell type-specific and/or organ-specific EVs, in particular, brain-specific and/or neuron-specific EVs, can be useful to enable a better diagnosis, prognosis, or monitoring of diseases, such as neurodegenerative diseases, as well as for improved prediction of treatment outcomes.

The present invention provides biomarkers for cell type-specific and/or organ-specific EV markers, e.g., brain-specific and/or neuron-specific EVs. Biomarkers levels are determined in a biological sample obtained from a subject. The markers of the invention include, but are not limited to, one or more biomarkers selected from Tables 1-5, or any combination thereof. As used herein, the term “one or more biomarkers” or “at least one of” is intended to mean that one or more (e.g., 1, 2, 3, 4, 5, 6, 7, 8, 9, 10, 11, 12, 13, 14, 15, 16, 17, 18, 19, 20, or more) markers selected from Tables 1-5, or any combination thereof, are assayed. Methods, kits, and panels provided herein also include any combination of e.g., 1, 2, 3, 4, 5, 6, 7, 8, 9, 10, 11, 12, 13, 14, 15, 16, 17, 18, 19, 20, or more markers selected from Tables 1-5, or any combination thereof.

In one embodiment, the biomarkers for brain-specific EVs include one or more biomarkers in Tables 1-5, or any combinations thereof.

In one embodiment, the biomarkers for neuron-specific EVs include one or more biomarkers in Tables 1 and 5, or any combinations thereof. In one embodiment, the biomarkers for neuron-specific EVs include one or more biomarker selected from the group consisting of GABRG2, SVOP, SLC32A1, GRM7, GABRB3, CHRNB2, SLC12A5, GRM8, PTPRT, SLC6A17, PCDHAC2, PLPPR4, C11orf87, SORCS3, CALY, PTPRR, KIAA1549L, HCN1, CDH18, TMEM132D, GPR158, FRRS1L, ATP2B3, GRIA4, ST8SIA3, HS6ST3, SEZ6, and NRXN3.

In one embodiment, the biomarkers for astrocyte-specific EVs include one or more biomarkers in Table 2, or any combinations thereof.

In one embodiment, the biomarkers for oligodendrocyte-specific EVs include one or more biomarkers in Table 3, or any combinations thereof.

In one embodiment, the biomarkers for microglial-specific EVs include one or more biomarkers in Table 4, or any combinations thereof.

TABLE 1
Biomarkers for Neuron-specific EVs

| Gene | Gene Name | Accession | Gene ID |
|---|---|---|---|
| GABRG2 | gamma-aminobutyric acid type A receptor | NM_198904.4 | 2566 |
| | subunit gamma2 | | |
| GABRB2 | gamma-aminobutyric acid type A receptor | NM_021911.3 | 2561 |
| | subunit beta2 | | |
| CHRNB2 | cholinergic receptor nicotinic beta 2 subunit | NM_000748.3 | 1141 |
| GABRA4 | gamma-aminobutyric acid type A receptor | NM_000809.4 | 2557 |
| | subunit alpha4 | | |
| ADGRA1 | adhesion G protein-coupled receptor A1 | NM_001083909.3 | 84435 |
| SYNDIG1L | synapse differentiation inducing 1 like | NM_001105579.2 | 646658 |
| CTXN2 | cortexin 2 | NM_001145668.2 | 399697 |
| SMIM43 | small integral membrane protein 43 | NM_001384332.1 | 132332 |
| CACNG3 | calcium voltage-gated channel auxiliary | NM_006539.4 | 10368 |
| | subunit gamma 3 | | |
| JPH3 | junctophilin 3 | NM_020655.4 | 57338 |
| TMEM266 | transmembrane protein 266 | NM_152335.3 | 123591 |
| GRM7 | glutamate metabotropic receptor 7 | NM_000844.4 | 2917 |
| MC4R | melanocortin 4 receptor | NM_005912.3 | 4160 |
| SYNPR | synaptoporin | NM_001130003.2 | 132204 |
| GPR137C | G protein-coupled receptor 137C | NM_001099652.2 | 283554 |
| SLC38A8 | solute carrier family 38 member 8 | NM_001080442.3 | 146167 |
| LRRC55 | leucine rich repeat containing 55 | NM_001005210.4 | 219527 |
| TMEM151B | transmembrane protein 151B | NM_001137560.2 | 441151 |
| SYNGR3 | synaptogyrin 3 | NM_004209.6 | 9143 |
| ZDHHC22 | zinc finger DHHC-type palmitoyltransferase | NM_174976.2 | 283576 |
| | 22 | | |
| DRD1 | dopamine receptor D1 | NM_000794.5 | 1812 |
| PCDH11Y | protocadherin 11 Y-linked | NM_032973.2 | 83259 |
| CACNG2 | calcium voltage-gated channel auxiliary | NM_006078.5 | 10369 |
| | subunit gamma 2 | | |
| OPRK1 | opioid receptor kappa 1 | NM_000912.5 | 4986 |
| GLRA2 | glycine receptor alpha 2 | NM_002063.4 | 2742 |
| SVOP | SV2 related protein | NM_018711.5 | 55530 |
| GPR149 | G protein-coupled receptor 149 | NM_001038705.3 | 344758 |
| HTR5A | 5-hydroxytryptamine receptor 5A | NM_024012.4 | 3361 |
| GABRA5 | gamma-aminobutyric acid type A receptor | NM_000810.4 | 2558 |
| | subunit alpha5 | | |
| GPR151 | G protein-coupled receptor 151 | NM_194251.3 | 134391 |
| GPR6 | G protein-coupled receptor 6 | NM_005284.5 | 2830 |
| GPR83 | G protein-coupled receptor 83 | NM_016540.4 | 10888 |
| TRPC5 | transient receptor potential cation channel | NM_012471.3 | 7224 |
| | subfamily C member 5 | | |
| KCNH5 | potassium voltage-gated channel subfamily H | NM_139318.5 | 27133 |
| | member 5 | | |
| SLC7A14 | solute carrier family 7 member 14 | NM_020949.3 | 57709 |
| GLRA3 | glycine receptor alpha 3 | NM_006529.4 | 8001 |
| SCN1A | sodium voltage-gated channel alpha subunit 1 | NM_001165963.4 | 6323 |
| OR1F1 | olfactory receptor family 1 subfamily F | NM_001370640.3 | 4992 |
| | member 1 | | |
| TMEM196 | transmembrane protein 196 | NM_001363562.2 | 256130 |
| GABRG3 | gamma-aminobutyric acid type A receptor | NM_033223.5 | 2567 |
| | subunit gamma 3 | | |
| SLC35F4 | solute carrier family 35 member F4 | NM_001306087.2 | 341880 |
| SLC32A1 | solute carrier family 32 member 1 | NM_080552.3 | 140679 |
| KCNJ6 | potassium inwardly rectifying channel | NM_002240.5 | 3763 |
| | subfamily J member 6 | | |
| KCNC1 | potassium voltage-gated channel subfamily C | NM_001112741.2 | 3746 |
| | member 1 | | |
| CALN1 | calneuron 1 | NM_031468.4 | 83698 |
| KCND2 | potassium voltage-gated channel subfamily D | NM_012281.3 | 3751 |
| | member 2 | | |
| KCNV1 | potassium voltage-gated channel modifier | NM_014379.4 | 27012 |
| | subfamily V member 1 | | |
| GABRA6 | gamma-aminobutyric acid type A receptor | NM_000811.3 | 2559 |
| | subunit alpha 6 | | |
| LHFPL4 | LHFPL tetraspan subfamily member 4 | NM_198560.3 | 375323 |
| PTPN5 | protein tyrosine phosphatase non-receptor | NM_006906.2 | 84867 |
| | type 5 | | |
| ASIC2 | acid sensing ion channel subunit 2 | NM_183377.2 | 40 |
| FAM155A | Family With Sequence Similarity 155 | NM_001080396.3 | 728215 |
| | Member A | | |
| RNF175 | ring finger protein 175 | NM_173662.4 | 285533 |
| FAM189A1 | family with sequence similarity 189 member | NM_015307.2 | 23359 |
| | A1 | | |
| KCNA1 | potassium voltage-gated channel subfamily A | NM_000217.3 | 3736 |
| | member 1 | | |
| GABRB3 | gamma-aminobutyric acid type A receptor | NM_000814.6 | 2562 |
| | subunit beta3 | | |
| MCHR2 | melanin concentrating hormone receptor 2 | NM_001040179.2 | 84539 |
| JPH4 | junctophilin 4 | NM_001146028.2 | 84502 |
| GPR26 | G protein-coupled receptor 26 | NM_153442.4 | 2849 |
| UNC80 | unc-80 homolog, NALCN channel complex | NM_001371986.1 | 285175 |
| | subunit | | |
| GPR22 | G protein-coupled receptor 22 | NM_005295.3 | 2845 |
| SLC30A3 | solute carrier family 30 member 3 | NM_003459.5 | 7781 |
| GRIN2A | glutamate ionotropic receptor NMDA type | NM_001134407.3 | 2903 |
| | subunit 2A | | |
| VSTM5 | V-set and transmembrane domain containing | NM_001144871.2 | 387804 |
| | 5 | | |
| GRIK1 | glutamate ionotropic receptor kainate type | NM_001330994.2 | 2897 |
| | subunit 1 | | |
| KCNC2 | potassium voltage-gated channel subfamily C | NM_139137.4 | 3747 |
| | member 2 | | |
| KCNH1 | potassium voltage-gated channel subfamily H | NM_172362.3 | 3756 |
| | member 1 | | |
| SLC1A6 | solute carrier family 1 member 6 | NM_005071.3 | 6511 |
| LRRTM3 | leucine rich repeat transmembrane neuronal 3 | NM_178011.5 | 347731 |
| SCN8A | sodium voltage-gated channel alpha subunit 8 | NM_001330260.2 | 6334 |
| NDST3 | N-deacetylase and N-sulfotransferase 3 | NM_004784.3 | 9348 |
| PLPPR5 | phospholipid phosphatase related 5 | NM_001037317.2 | 163404 |
| SLC10A4 | solute carrier family 10 member 4 | NM_152679.4 | 201780 |
| SLC35F3 | solute carrier family 35 member F3 | NM_173508.4 | 148641 |
| SYT4 | synaptotagmin 4 | NM_020783.4 | 6860 |
| GPR52 | G protein-coupled receptor 52 | NM_005684.5 | 9293 |
| TMEM145 | transmembrane protein 145 | NM_173633.3 | 284339 |
| CNIH2 | cornichon family AMPA receptor auxiliary | NM_182553.3 | 254263 |
| | protein 2 | | |
| KCNJ3 | potassium inwardly rectifying channel | NM_002239.4 | 3760 |
| | subfamily J member 3 | | |
| SYT13 | synaptotagmin 13 | NM_020826.3 | 57586 |
| GABBR2 | gamma-aminobutyric acid type B receptor | NM_005458.8 | 9568 |
| | subunit 2 | | |
| SLC17A7 | solute carrier family 17 member 7 | NM_020309.4 | 57030 |
| ANO3 | anoctamin 3 | NM_031418.4 | 63982 |
| CNNM1 | cyclin and CBS domain divalent metal cation | NM_020348.3 | 26507 |
| | transport mediator 1 | | |
| GPR88 | G protein-coupled receptor 88 | NM_022049.3 | 54112 |
| SLC6A17 | solute carrier family 6 member 17 | NM_001010898.4 | 388662 |
| CACNG8 | calcium voltage-gated channel auxiliary | NM_031895.6 | 59283 |
| | subunit gamma 8 | | |
| ALK | ALK receptor tyrosine kinase | NM_004304.5 | 238 |
| LRRTM1 | leucine rich repeat transmembrane neuronal 1 | NM_178839.5 | 347730 |
| GPR12 | G protein-coupled receptor 12 | NM_005288.4 | 2835 |
| SUN3 | Sad1 and UNC84 domain containing 3 | NM_001030019.2 | 256979 |
| SCN2A | sodium voltage-gated channel alpha subunit 2 | NM_001040142.2 | 6326 |
| GRIN2B | glutamate ionotropic receptor NMDA type | NM_000834.5 | 2904 |
| | subunit 2B | | |
| PCDHA1 | protocadherin alpha 1 | NM_018900.4 | 56147 |
| GABRA1 | gamma-aminobutyric acid type A receptor | NM_001127644.2 | 2554 |
| | subunit alpha1 | | |
| CABP7 | calcium binding protein 7 | NM_182527.3 | 164633 |
| SHISA9 | shisa family member 9 | NM_001145204.3 | 729993 |
| HS3ST5 | heparan sulfate-glucosamine 3- | NM_153612.4 | 222537 |
| | sulfotransferase 5 | | |
| GRIA1 | lutamate ionotropic receptor AMPA type | NM_000827.4 | 2890 |
| | subunit 1 | | |
| SV2C | synaptic vesicle glycoprotein 2C | NM_014979.4 | 22987 |
| GRIK2 | glutamate ionotropic receptor kainate type | NM_021956.5 | 2898 |
| | subunit 2 | | |
| CSMD3 | CUB and Sushi multiple domains 3 | NM_198123.2 | 114788 |
| SOGA3 | SOGA family member 3 | NM_001400265.1 | 387104 |
| TMEM63C | transmembrane protein 63C | NM_020431.4 | 57156 |
| SLC17A6 | solute carrier family 17 member 6 | NM_020346.3 | 57084 |
| ADCY1 | adenylate cyclase 1 | NM_021116.4 | 107 |
| CACNA1E | calcium voltage-gated channel subunit alpha1E | NM_001205293.3 | 777 |
| GRM5 | glutamate metabotropic receptor 5 | NM_001143831.3 | 2915 |
| SV2B | synaptic vesicle glycoprotein 2B | NM_001323032.3 | 9899 |
| GRIA3 | glutamate ionotropic receptor AMPA type | NM_007325.5 | 2892 |
| | subunit 3 | | |
| WSCD2 | WSC domain containing 2 | NM_014653.4 | 9671 |
| SCN3A | sodium voltage-gated channel alpha subunit 3 | NM_006922.4 | 6328 |
| FNDC9 | fibronectin type III domain containing 9 | NM_001001343.4 | 408263 |
| HTR2C | 5-hydroxytryptamine receptor 2C | NM_000868.4 | 3358 |
| GRM8 | glutamate metabotropic receptor 8 | NM_000845.3 | 2918 |
| CDH7 | cadherin 7 | NM_004361.5 | 1005 |
| DISP2 | dispatched RND transporter family member 2 | NM_033510.3 | 85455 |
| PLPPR4 | phospholipid phosphatase related 4 | NM_014839.5 | 9890 |
| KIRREL3 | kirre like nephrin family adhesion molecule 3 | NM_032531.4 | 84623 |
| ATP8A2 | ATPase phospholipid transporting 8A2 | NM_016529.6 | 51761 |
| GRM1 | glutamate metabotropic receptor 1 | NM_001278064.2 | 2911 |
| CSMD1 | CUB and Sushi multiple domains 1 | NM_033225.6 | 64478 |
| PCDHGC4 | protocadherin gamma subfamily C, 4 | NM_018928.3 | 56098 |
| CDH9 | cadherin 9 | NM_016279.4 | 1007 |
| LRTM2 | leucine rich repeats and transmembrane | NM_001039029.3 | 654429 |
| | domains 2 | | |
| SLC12A5 | solute carrier family 12 member 5 | NM_020708.5 | 57468 |
| SLC4A10 | solute carrier family 4 member 10 | NM_001178015.2 | 57282 |
| CELSR3 | cadherin EGF LAG seven-pass G-type | NM_001407.3 | 1951 |
| | receptor 3 | | |
| CDH8 | cadherin 8 | NM_001796.5 | 1006 |
| C11orf87 | chromosome 11 open reading frame 87 | NM_207645.4 | 399947 |
| SCN3B | sodium voltage-gated channel beta subunit 3 | NM_001040151.2 | 55800 |
| NETO1 | neuropilin and tolloid like 1 | NM_138966.5 | 81832 |
| TMEM130 | transmembrane protein 130 | NM_152913.3 | 222865 |
| LAMP5 | lysosomal associated membrane protein | NM_012261.4 | 24141 |
| | family member 5 | | |
| ATP1A3 | ATPase Na+/K+ transporting subunit alpha 3 | NM_152296.5 | 478 |
| PTPRT | protein tyrosine phosphatase receptor type T | NM_007050.6 | 11122 |
| CACNA1B | calcium voltage-gated channel subunit alpha1B | NM_000718.4 | 774 |
| UNC5D | unc-5 netrin receptor D | NM_080872.4 | 137970 |
| SYP | synaptophysin | NM_003179.3 | 6855 |
| SLITRK5 | SLIT and NTRK like family member 5 | NM_001384609.1 | 26050 |
| SORCS3 | sortilin related VPS10 domain containing | NM_014978.3 | 22986 |
| | receptor 3 | | |
| SYT2 | synaptotagmin 2 | NM_177402.5 | 127833 |
| B4GALNT1 | beta-1,4-N-acetyl-galactosaminyltransferase | NM_001478.5 | 2583 |
| | 1 | | |
| SCN2B | sodium voltage-gated channel beta subunit 2 | NM_004588.5 | 6327 |
| PCDH8 | protocadherin 8 | NM_002590.4 | 5100 |
| SLC8A2 | solute carrier family 8 member A2 | NM_015063.3 | 6543 |
| CALY | calcyon neuron specific vesicular protein | NM_015722.4 | 50632 |
| PTPRR | protein tyrosine phosphatase receptor type R | NM_002849.4 | 5801 |
| KIAA1549L | KIAA1549 like | NM_012194.3 | 25758 |
| ST8SIA5 | ST8 alpha-N-acetyl-neuraminide alpha-2,8- | NM_013305.6 | 29906 |
| | sialyltransferase 5 | | |
| HCN1 | hyperpolarization activated cyclic nucleotide | NM_021072.4 | 348980 |
| | gated potassium channel 1 | | |
| SYT1 | synaptotagmin 1 | NM_005639.3 | 6857 |
| CDH18 | cadherin 18 | NM_004934.5 | 1016 |
| GPR158 | G protein-coupled receptor 158 | NM_020752.3 | 57512 |
| FRRS1L | ferric chelate reductase 1 like | NM_014334.4 | 23732 |
| ATP2B2 | ATPase plasma membrane Ca2+ transporting | NM_001001331.4 | 491 |
| | 2 | | |
| EPHA5 | EPH receptor A5 | NM_001281766.3 | 2044 |
| CNTNAP5 | contactin associated protein family member 5 | NM_001367498.1 | 129684 |
| TMEM132D | transmembrane protein 132D | NM_133448.3 | 121256 |
| CNTNAP2 | contactin associated protein 2 | NM_014141.6 | 26047 |
| GRIA4 | glutamate ionotropic receptor AMPA type | NM_000829.4 | 2893 |
| | subunit 4 | | |
| PCDHAC2 | protocadherin alpha subfamily C, 2 | NM_018899.6 | 56134 |
| ST8SIA3 | ST8 alpha-N-acetyl-neuraminide alpha-2,8- | NM_015879.3 | 51046 |
| | sialyltransferase 3 | | |
| HS6ST3 | heparan sulfate 6-O-sulfotransferase 3 | NM_153456.4 | 266722 |
| ATP2B3 | ATPase plasma membrane Ca2+ transporting | NM_001001344.3 | 492 |
| | 3 | | |
| DCC | DCC netrin 1 receptor | NM_005215.4 | 1630 |
| NRXN3 | neurexin 3 | NM_001330195.2 | 9369 |
| SEZ6 | seizure related 6 homolog | NM_178860.5 | 124925 |

TABLE 2
Biomarkers for Astrocyte-specific EV

| Gene | Gene Name | Accession | GeneID |
|---|---|---|---|
| ELOVL2 | ELOVL fatty acid elongase 2 | NM_017770.4 | 54898 |
| NKAIN4 | sodium/potassium transporting ATPase | NM_152864.4 | 128414 |
| | interacting 4 | | |
| KCNQ2 | potassium voltage-gated channel subfamily | NM_172107.4 | 3785 |
| | Q member 2 | | |
| CERS1 | ceramide synthase 1 | NM_021267.5 | 10715 |
| CYP46A1 | cytochrome P450 family 46 subfamily A | NM_006668.2 | 10858 |
| | member 1 | | |
| ADCY8 | adenylate cyclase 8 | NM_001115.3 | 114 |
| RGR | retinal G protein coupled receptor | NM_001012720.2 | 5995 |
| NTSR2 | neurotensin receptor 2 | NM_012344.4 | 23620 |
| MMD2 | monocyte to macrophage differentiation- | NM_175217.6 | 75104 |
| | associated 2 | | |
| GRIN2C | glutamate ionotropic receptor NMDA type | NM_000835.6 | 2905 |
| | subunit 2C | | |
| CRB1 | crumbs cell polarity complex component 1 | NM_001193640.2 | 23418 |
| SLC25A48 | solute carrier family 25 member 48 | NM_001349336.2 | 153328 |
| SLCO1C1 | solute carrier organic anion transporter | NM_017435.5 | 53919 |
| | family member 1C1 | | |
| PCDHA11 | protocadherin alpha 11 | NM_018902.5 | 56138 |
| ADORA1 | adenosine A1 receptor | NM_000674.3 | 134 |
| KCNE5 | potassium voltage-gated channel subfamily E | NM_012282.4 | 23630 |
| | regulatory subunit 5 | | |
| TRPM3 | transient receptor potential cation channel | NM_001366145.2 | 80036 |
| | subfamily M member 3 | | |
| LCTL | lactase like | NM_207338.4 | 197021 |
| NAT8L | N-acetyltransferase 8 like | NM_178557.4 | 339983 |
| GABRG1 | gamma-aminobutyric acid type A receptor | NM_173536.4 | 2565 |
| | subunit gamma1 | | |
| GABRA2 | gamma-aminobutyric acid type A receptor | NM_000807.4 | 2555 |
| | subunit alpha2 | | |
| RNF182 | ring finger protein 182 | NM_152737.4 | 221687 |
| PCDHA13 | protocadherin alpha 13 | NM_018904.3 | 56136 |
| LRRC3B | leucine rich repeat containing 3B | NM_001317811.2 | 116135 |
| MLC1 | modulator of VRAC current 1 | NM_015166.4 | 23209 |
| TMEM229A | transmembrane protein 229A | NM_001136002.2 | 730130 |
| TTYH1 | tweety family member 1 | NM_020659.4 | 57348 |
| PSD2 | pleckstrin and Sec7 domain containing 2 | NM_032289.4 | 84249 |
| SLC25A18 | solute carrier family 25 member 18 | NM_031481.3 | 83733 |
| SLC6A11 | solute carrier family 6 member 11 | NM_014229.3 | 6538 |
| CDH20 | cadherin 20 | NM_031891.4 | 28316 |
| DIPK1C | divergent protein kinase domain 1C | NM_001044369.3 | 125704 |
| ADGRB1 | adhesion G protein-coupled receptor B1 | NM_001702.3 | 575 |
| GPR37L1 | G protein-coupled receptor 37 like 1 | NM_004767.5 | 9283 |
| HEPACAM | hepatic and glial cell adhesion molecule | NM_152722.5 | 220296 |
| KIAA0319 | KIAA0319 | NM_014809.4 | 9856 |
| MEGF10 | multiple EGF like domains 10 | NM_001256545.2 | 84466 |
| SLC39A12 | solute carrier family 39 member 12 | NM_001145195.2 | 221074 |

TABLE 3
Biomarkers for Oligodendrocyte -specific EVs

| Gene | Gene Name | Accession | Gene ID |
|---|---|---|---|
| SLC5A11 | solute carrier family 5 member 11 | NM_001352248.3 | 115584 |
| LHFPL3 | LHFPL tetraspan subfamily member 3 | NM_199000.3 | 375612 |
| TMEM235 | transmembrane protein 235 | NM_001204210.2 | 283999 |
| CACNG5 | calcium voltage-gated channel auxiliary | NM_145811.3 | 27091 |
| | subunit gamma 5 | | |
| OR9A2 | olfactory receptor family 9 subfamily A | NM_001001658.1 | 135924 |
| | member 2 | | |
| GPR62 | G protein-coupled receptor 62 | NM_080865.4 | 118442 |
| PLPPR1 | phospholipid phosphatase related 1 | NM_207299.2 | 54886 |
| PCDHA5 | protocadherin alpha 5 | NM_018908.3 | 56143 |
| KCNK10 | potassium two pore domain channel | NM_138317.3 | 54207 |
| | subfamily K member 10 | | |
| KCNH8 | potassium voltage-gated channel subfamily | NM_144633.3 | 131096 |
| | H member 8 | | |
| CACNG4 | calcium voltage-gated channel auxiliary | NM_014405.4 | 27092 |
| | subunit gamma 4 | | |
| GALR1 | galanin receptor 1 | NM_001480.4 | 2587 |
| PCDH15 | protocadherin related 15 | NM_001384140.1 | 65217 |
| UGT8 | UDP glycosyltransferase 8 | NM_001128174.3 | 7368 |
| PLPP4 | phospholipid phosphatase 4 | NM_001030059.3 | 196051 |
| OPALIN | oligodendrocytic myelin paranodal and | NM_033207.5 | 93377 |
| | inner loop protein | | |
| MEGF11 | multiple EGF like domains 11 | NM_001385028.1 | 84465 |
| CMTM5 | CKLF like MARVEL transmembrane | NM_001288746.2 | 116173 |
| | domain containing 5 | | |
| MOG | myelin oligodendrocyte glycoprotein | NM_206809.4 | 4340 |
| MAG | myelin associated glycoprotein | NM_002361.4 | 4099 |
| GPR37 | G protein-coupled receptor 37 | NM_005302.5 | 2861 |
| CA14 | carbonic anhydrase 14 | NM_012113.3 | 23632 |

TABLE 4
Biomarkers for Microglial -specific EVs

| Gene | Gene Name | Accession | Gene ID |
|---|---|---|---|
| SYNDIG1 | synapse differentiation | NM_024893.3 | 79953 |
| | inducing 1 | | |
| HS3ST4 | heparan sulfate-glucosamine 3- | NM_006040.3 | 9951 |
| | sulfotransferase 4 | | |
| CLEC9A | C-type lectin domain | NM_207345.4 | 283420 |
| | containing 9A | | |

TABLE 5
Biomarkers for Neuron-specific EVs

| Gene | Gene Name | Accession | GeneID |
|---|---|---|---|
| GABRG2 | gamma-aminobutyric acid type A receptor | NM_198904.4 | 2566 |
| | subunit gamma2 | | |
| SVOP | SV2 related protein | NM_018711.5 | 55530 |
| SLC32A1 | solute carrier family 32 member 1 | NM_080552.3 | 140679 |
| GRM7 | glutamate metabotropic receptor 7 | NM_000844.4 | 2917 |
| GABRB3 | gamma-aminobutyric acid type A receptor | NM_000814.6 | 2562 |
| | subunit beta3 | | |
| CHRNB2 | cholinergic receptor nicotinic beta 2 subunit | NM_000748.3 | 1141 |
| SLC12A5 | solute carrier family 12 member 5 | NM_020708.5 | 57468 |
| GRM8 | glutamate metabotropic receptor 8 | NM_000845.3 | 2918 |
| PTPRT | protein tyrosine phosphatase receptor type T | NM_007050.6 | 11122 |
| SLC6A17 | solute carrier family 6 member 17 | NM_001010898.4 | 388662 |
| PCDHAC2 | protocadherin alpha subfamily C, 2 | NM_018899.6 | 56134 |
| PLPPR4 | phospholipid phosphatase related 4 | NM_014839.5 | 9890 |
| C11orf87 | chromosome 11 open reading frame 87 | NM_207645.4 | 399947 |
| SORCS3 | sortilin related VPS10 domain containing | NM_014978.3 | 22986 |
| | receptor 3 | | |
| CALY | Calcyon Neuron Specific Vesicular Protein | NM_015722.4 | 50632 |
| PTPRR | protein tyrosine phosphatase receptor type | NM_002849.4 | 5801 |
| | R | | |
| KIAA1549L | KIAA1549 like | NM_012194.3 | 25758 |
| HCN1 | hyperpolarization activated cyclic | NM_021072.4 | 348980 |
| | nucleotide gated potassium channel 1 | | |
| CDH18 | cadherin 18 | NM_004934.5 | 1016 |
| TMEM132D | transmembrane protein 132D | NM_133448.3 | 121256 |
| GPR158 | G protein-coupled receptor 158 | NM_020752.3 | 57512 |
| FRRS1L | ferric chelate reductase 1 like | NM_014334.4 | 23732 |
| ATP2B2 | ATPase plasma membrane Ca2+ | NM_001001331.4 | 491 |
| | transporting 2 | | |
| GRIA4 | glutamate ionotropic receptor AMPA type | NM_000829.4 | 2893 |
| | subunit 4 | | |
| ST8SIA3 | ST8 alpha-N-acetyl-neuraminide alpha-2,8- | NM_015879.3 | 51046 |
| | sialyltransferase 3 | | |
| HS6ST3 | heparan sulfate 6-O-sulfotransferase 3 | NM_153456.4 | 266722 |
| SEZ6 | seizure related 6 homolog | NM_178860.5 | 124925 |
| NRXN3 | neurexin 3 | NM_001330195.2 | 9369 |

Each GenBank number is incorporated herein by reference in the version available on the filing date of the application to which this application claims priority.

The levels of the biomarkers of the present invention may be determined by any suitable means, methods or techniques known in the art.

In some embodiments, with regard to polypeptides or protein biomarkers, immunoassay devices and methods are often used. These devices and methods can utilize labeled molecules in various sandwich, competitive, or non-competitive assay formats, to generate a signal that is related to the presence or amount of a marker of interest. Additionally, certain methods and devices, such as biosensors and optical immunoassays, may be employed to determine the presence or amount of markers without the need for a labeled molecule.

In certain embodiments, the detection method is an immunodetection method involving an antibody that specifically binds to one or more of the markers in Tables 1-5. The steps of various useful immunodetection methods have been described in the scientific literature, such as, e.g., Nakamura et al. (1987), which is incorporated herein by reference. In general, the immunobinding methods include obtaining a sample suspected of containing a biomarker protein, peptide or antibody, and contacting the sample with an antibody or protein or peptide in accordance with the present invention, as the case may be, under conditions effective to allow the formation of immunocomplexes.

Detection of immunocomplex formation is well known in the art and may be achieved through the application of numerous approaches. These methods are generally based upon the detection of a label or marker, such as any radioactive, fluorescent, biological or enzymatic tags or labels of standard use in the art. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art. The protein employed in the detection may itself be linked to a detectable label, wherein one would then simply detect this label, thereby allowing the amount of the primary immune complexes in the composition to be determined. Alternatively, the first added component that becomes bound within the primary immune complexes may be detected by means of a second binding ligand that has binding affinity for the encoded protein, peptide or corresponding antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody, that has binding affinity for the encoded protein, peptide or corresponding antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

Any suitable immunoassay may be utilized, for example, enzyme-linked immunoassays (ELISA), radioimmunoassay (RIAs), competitive binding assays, planar waveguide technology, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like. In some embodiment, the biomarkers of the invention can be identified by other techniques such as Western blotting, dot blotting, and FACS analyses.

The biomarkers of the invention can also be measured, quantitated, detected, and otherwise analyzed using protein mass spectrometry methods and instrumentation. Protein mass spectrometry refers to the application of mass spectrometry to the study of proteins. Although not intending to be limiting, two approaches are typically used for characterizing proteins using mass spectrometry. In the first, intact proteins are ionized and then introduced to a mass analyzer. This approach is referred to as “top-down” strategy of protein analysis. The two primary methods for ionization of whole proteins are electrospray ionization (ESI) and matrix-assisted laser desorption/ionization (MALDI). In the second approach, proteins are enzymatically digested into smaller peptides using a protease such as trypsin. Subsequently these peptides are introduced into the mass spectrometer and identified by peptide mass fingerprinting or tandem mass spectrometry. Hence, this latter approach (also called “bottom-up” proteomics) uses identification at the peptide level to infer the existence of proteins.

The protein biomarkers of the invention can also be measured in complex mixtures of proteins and molecules that co-exist in a biological medium or sample, however, fractionation of the sample may be required and is contemplated herein. It will be appreciated that ionization of complex mixtures of proteins can result in situation where the more abundant proteins have a tendency to “drown” or suppress signals from less abundant proteins in the same sample. In addition, the mass spectrum from a complex mixture can be difficult to interpret because of the overwhelming number of mixture components. Fractionation can be used to first separate any complex mixture of proteins prior to mass spectrometry analysis. Two methods are widely used to fractionate proteins, or their peptide products from an enzymatic digestion. The first method fractionates whole proteins and is called two-dimensional gel electrophoresis. The second method, high performance liquid chromatography (LC or HPLC) is used to fractionate peptides after enzymatic digestion. In some situations, it may be desirable to combine both of these techniques. Any other suitable methods known in the art for fractionating protein mixtures are also contemplated herein.

Characterization of protein mixtures using HPLC/MS may also be referred to in the art as “shotgun proteomics” and MuDPIT (Multi-Dimensional Protein Identification Technology). A peptide mixture that results from digestion of a protein mixture is fractionated by one or two steps of liquid chromatography (LC). The eluent from the chromatography stage can be either directly introduced to the mass spectrometer through electrospray ionization, or laid down on a series of small spots for later mass analysis using MALDI.

In certain embodiments, the invention involves the detection of nucleic acid biomarkers, e.g., the corresponding genes or mRNA of the protein markers of the invention.

In various embodiments, the methods of the present invention generally involve the determination of expression levels of a set of genes in extracellular vesicles isolated from a biological sample. Determination of gene expression levels in the practice of the inventive methods may be performed by any suitable method. For example, determination of gene expression levels may be performed by detecting the expression of mRNA expressed from the genes of interest and/or by detecting the expression of a polypeptide encoded by the genes by PCR, e.g., reverse transcription polymerase chain reaction (RT-PCR), quantitative reverse transcription polymerase chain reaction (qRT-PCR), RNA sequencing (RNA seq), or array analysis. In some embodiments, the expression level of the biomarker is determined by measuring the mRNA or miRNA level of the biomarker.

The analysis of a plurality of markers may be carried out separately or simultaneously with one test sample. Several markers may be combined into one test for efficient processing of a multiple of samples. In addition, one skilled in the art would recognize the value of testing multiple samples (for example, at successive time points) from the same individual. Such testing of serial samples will allow the identification of changes in marker levels over time. Increases or decreases in marker levels, as well as the absence of change in marker levels, would provide useful information about the disease status that includes, but is not limited to identifying the approximate time from onset of the event, the appropriateness of drug therapies, the effectiveness of various therapies, identification of the severity of the event, identification of the disease severity, and identification of the patient's outcome, including risk of future events.

An assay consisting of a combination of the markers referenced in the instant invention may be constructed to provide relevant information related to differential diagnosis. Such a panel may be constructed using 1, 2, 3, 4, 5, 6, 7, 8, 9, 10, 15, 20, or more or individual markers. The analysis of a single marker or subsets of markers comprising a larger panel of markers could be carried out methods described within the instant invention to optimize clinical sensitivity or specificity in various clinical settings.

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings. Particularly useful physical formats comprise surfaces having a plurality of discrete, addressable locations for the detection of a plurality of different markers. Such formats include protein microarrays, or “protein chips” and capillary devices.

D. Methods for Purification, Isolation and/or Enrichment of Extracellular Vesicles

The present invention provides methods for the purification of extracellular vesicles (EVs), and methods for isolation and/or enrichment of cell type-specific and/or organ-specific EVs, e.g., brain- and/or neuron-specific EVs, from a sample, e.g., a biological sample such as plasma or cerebrospinal fluid.

Suitable methods of purifying or isolating EVs are known in the art, including, but not limited to, differential centrifugation, anion exchange and/or gel permeation chromatography, sucrose density gradients or organelle electrophoresis, magnetic activated cell sorting (MACS), nanomembrane ultrafiltration concentrator (see, e.g., U.S. Pat. Nos. 6,899,863 and 6,812,023. U.S. Pat. No. 7,198,923; Taylor and Gercel-Taylor, 2008; Cheruvanky et al., 2007). In some embodiments, EVs can also be identified and isolated from bodily fluid of a subject by a microchip technology that uses a unique microfluidic platform to efficiently and selectively separate tumor-derived microvesicles. Each of the foregoing references is incorporated by reference herein for its teaching of these methods.

In some embodiments, the EVs can be purified from a sample using a mixed mode resin (MMR) or size exclusion chromatography (SEC), as described herein.

Purification of EVs with Mixed Mode Resin

Mixed mode resins (MMR) can be used for purification of the EVs from a sample, e.g., a biological sample, from a subject. An exemplary mixed mode resin is Capto™ Core 700. This resin comprises beads with an inert outer shell and pores that exclude molecules larger than 700 kDa. MMR beads have a core that contains octylamine ligands that are both hydrophobic and positively charged, efficiently trapping proteins that enter the beads.

The MMR beads can “trap” contaminants within the bead, thus enabling purification of extracellular vesicles from a sample, e.g., a biological sample, e.g., a plasma or a CSF sample. The disclosed methods are independent of columns and other chromatography equipment and are compatible with high-throughput purification of extracellular vesicles.

In some embodiments, the MMR beads exclude molecules which are greater than the size of a target EV, e.g., greater than about 400, 500, 600, or 700 kDa. In some embodiments, the beads comprise an inactive bead exterior or shell. In some embodiments, the shell comprises pores. In some embodiments, the beads contain a ligand-activated core such as an octylamine ligand. In some embodiments, the size-exclusion beads comprise a bind-elute resin. In some embodiments, the size-exclusion beads are Capto™ Core resin beads, e.g., Capto™ Core 700 resin beads or Capto™ Core resin 400 beads.

In some embodiments, the method does not require use of columns for purification of EVs from biological samples.

In some aspects, the disclosure provides methods for purifying extracellular vesicles from a biological sample comprising combining a liquid biological sample containing extracellular vesicles with mixed mode resin (MMR) beads capable of capturing molecules smaller the size of a target EV, e.g., greater than about 700 kDa, to create a mixture, and separating and removing the MMR beads from the mixture, such that the extracellular vesicles remain, or by removing the supernatant from the mixture, thereby purifying the extracellular vesicles.

In some embodiments, the biological sample is obtained from a subject. In some embodiments, the biological sample is a liquid biological sample. One skilled in the art will recognize that a biological sample can be, but is not limited to, the following bodily fluids: peripheral blood, plasma, serum, cerebrospinal fluid (CSF), ascites, sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids. A biological sample may also include the blastocyst cavity, umbilical cord blood, or maternal circulation that may be of fetal or maternal origin. The biological sample may also be a tissue sample or biopsy, from which EVs may be obtained. In one embodiment, the biological sample is a brain tissue. In one embodiment, the biological sample is a plasma sample. In another embodiment, the biological sample is a CSF sample.

In some embodiments, the methods of the disclosure are performed as in-slurry methods. The MMR beads can be suspended in a buffer to create a slurry. In some embodiments, the MMR beads can be suspended in an equal volume of buffer to create a 50% slurry, or in any volume buffer effective to produce a slurry with an effective amount of MMR beads to purify EVs from a sample. For example, the slurry can be a 20%, 30%, 40%, 50%, 60%, 70%, 80%, or 90% slurry. Any effective buffer can be used to produce the slurry. In some embodiments, PBS buffer is used.

The MMR beads can be mixed with the biological sample in an amount of, for example, 20 μL, 25 μL, 50 μL, 75 μL, 100 μL, 150 μL, 200 μL, 250 μL, 300 μL, 350 μL, or 400 μL per 1 mL of liquid biological sample.

The combination, or mixture, of MMR beads and the sample containing the EVs can be mixed, agitated, or rotated prior to separation and removal of the MMR beads. For example, the mixture can be mixed, agitated, or rotated for about 10, 20, 30, 40, 45, 50, 55, 60, 65, 70, 75 or more minutes prior to separation and removal of the MMR beads. In some embodiments, the mixture is mixed, agitated, or rotated for between about 30 minutes and about 1 hour. In some embodiments, the mixture is mixed, agitated, or rotated for about 45 minutes. The mixture can be mixed, agitated, or rotated at room temperature, or at any temperature conducive to the capture of impurities in the sample by the MR beads.

The MMR beads can be separated from the mixture using any effective means known in the art. In some embodiments, centrifugation can be used to separate the MMR beads from the mixture. In some embodiments, the mixture is centrifuged at between about 600 g, 700 g, 800 g, 900 g or higher. In some embodiments, the mixture is centrifuged at about 800 g. In some embodiments, the mixture is centrifuged for enough time to adequately separate the MMR beads from remainder of the mixture to obtain the EVs in the supernatant. In some embodiments, the mixture is centrifuged for about 10 minutes, or for about 10, 15, 20, 25, 30, 40 or more minutes.

The MMR beads used in the methods of the disclosure can, in some embodiments, comprise an inactive bead exterior or shell. The exterior of the bead can comprise pores which allow molecules less than a certain size to pass through the exterior shell and be trapped in the core of the bead. In some embodiments, the beads can comprise a core comprising interior of the beads comprise a ligand such as a multimodal ligand such as octylamine ligand. In a preferred embodiment the total shell bead (i.e., shell plus core) thickness is preferably 40-100 microns in diameter, and the shell thickness is preferably 2-10 microns.

In some preferred embodiments, the MMR beads used in the methods of the disclosure comprise an inner porous core and an outer porous shell, wherein the inner core is provided with octylamine ligands and the shell is inactive, and wherein the porosity of the shell and core does not allow entering of molecules larger than about 700 kD.

In some embodiments, the MMR beads comprise a bind-elute resin. In some embodiments, the MMR beads are Capto™ Core bind-elute beads. In some embodiments, the MMR beads are Capto™ Core 700 bind-elute beads. Capto™ Core 700 chromatography resin (GE Healthcare Biosciences AB) comprise octylamine ligands within Capto™ Core 700 ‘beads’, and are designed to have both hydrophobic and positively charged properties that can trap molecules under 700 kilodaltons. Since extracellular vesicles exceed 700 kDa, and since the bead exterior is inactive, Capto Core 700 permits purification of extracellular vesicles by size exclusion. With standard gel filtration (size exclusion chromatography), molecules of smaller size spend more time penetrating pores of the stationary phase, and therefore exhibit higher retention (slower elusion) relative to larger molecules. In contrast, the ligand-activated pores of Capto™ Core 700 have electrostatic and hydrophobic interactions that “capture” molecules under 700 kDa.

In some embodiments, the biological sample, e.g., plasma or CSF, is subjected to a size exclusion chromatography (SEC) column prior to combining with the MMR beads capable of capturing molecules smaller than about 700 kDa. Any size exclusion chromatography resins known in the art are suitable for the methods of the present invention. In some embodiments, the size exclusion chromatography column comprises a stationary phase comprising a 6% cross-linked agarose size exclusion chromatography base matrix. In some embodiments, the 6% cross-linked agarose size exclusion chromatography base matrix is a Sepharose™ CL-6B resin.

In some embodiments, the biological sample, e.g., plasma or CSF, is further subjected to a cation exchange chromatography resin after the size exclusion chromatography column and prior to combining with the MMR beads capable of capturing molecules smaller than about 700 kDa. Any cation exchange chromatography resins known in the art are suitable for the methods of the present invention. Exemplary cation exchange chromatography resin include, but are not limited to, sulpfhydryl, sulfonate, sulfate, carboxymethyl, sulfoethyl, sulfopropyl, phosphate and sulfonate. In some embodiment, the cation exchange chromatography resin is Fractogel® EMD-SO3-resin.

The methods for EV purification disclosed herein can also be optimized for high-throughput applications.

Purification of EVs with Size Exclusion Chromatography

Size exclusion chromatography (SEC) can also be used for purification of EVs from a sample, e.g., a biological sample, from a subject. In some embodiments, EVs can be purified from a biological sample using size exclusion chromatography (SEC) columns with a stationary phase material comprising an agarose size exclusion chromatography base matrix, for example, a base matrix comprising 6% agarose content. In some embodiments, the stationary phase material is a 6% cross-linked agarose size exclusion chromatography base matrix. In some embodiments, the stationary phase material is a Sepharose™ resin, e.g., a Sepharose™ cross-linked resin such as Sepharose™ CL-6B resin. In SEC, a porous stationary phase is utilized to sort macromolecules and particulate matters according to their size. Components in a sample with small hydrodynamic radii are able to pass through the pores, thus resulting in late elution. Components with large hydrodynamic radii, including EVs, are excluded from entering the pores. The SEC columns used in the methods of the invention yield significant improvements in EV yield as compared to other columns at a fraction of the cost.

In some aspects, the disclosure provides methods for purifying extracellular vesicles from a biological sample, the method comprising providing a SEC column comprising a stationary phase material comprising a 6% cross-linked agarose size exclusion chromatography base matrix, e.g., a Sepharose™ CL-6B resin, introducing a sample comprising extracellular vesicles into the column, flowing the sample through the stationary phase material, and collecting fractions containing extracellular vesicles from the SEC column, thereby purifying the extracellular vesicles.

In some embodiments, the sample is a biological sample is obtained from a subject. In some embodiments, the biological sample is a liquid biological sample. One skilled in the art will recognize that a biological sample can be, but is not limited to, the following bodily fluids: peripheral blood, plasma, serum, cerebrospinal fluid (CSF), ascites, sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids. A biological sample may also include the blastocyst cavity, umbilical cord blood, or maternal circulation that may be of fetal or maternal origin. The biological sample may also be a tissue sample or biopsy, from which EVs may be obtained. In one embodiment, the biological sample is a brain tissue sample. In one embodiment, the biological sample is a plasma sample. In another embodiment, the liquid biological sample is a CSF sample.

In other embodiments, the SEC column is a 5 mL, 7 mL, 10 mL, 12 mL, 15 mL, 20 mL, or 25 mL volume column. In other embodiments, the SEC column is a 5 mL to 25 mL volume column, although columns outside of these ranges can also be used. In some embodiments, the SEC column is a 10 mL volume column. In other embodiments, the SEC column is a 20 mL volume column.

The SEC column comprising a 6% cross-linked agarose size exclusion chromatography base matrix, e.g., Sepharose™ CL-6B resin can be prepared by first washing the resin prior to addition to the column. In some embodiments, the 6% cross-linked agarose size exclusion chromatography base matrix, e.g., a Sepharose™ CL-6B resin is washed in buffer, e.g., PBS, prior to preparation of the column. The resin can be washed multiple times prior to preparation of the column. In some embodiments, the 6% cross-linked agarose size exclusion chromatography base matrix, e.g., Sepharose™ CL-6B resin is washed four or more times in buffer prior to preparation of the column.

Once the resin is washed, it can be added to a suitable column, e.g., a 10 mL or 20 mL column. In one embodiment, the SEC column comprises a housing having at least one wall defining a chamber having an entrance and an exit. In some embodiments, the stationary phase is washed prior to the column comprising the 6% cross-linked agarose size exclusion chromatography base matrix, e.g., Sepharose™ CL-6B resin is washed prior to introducing the sample comprising extracellular vesicles into the column. In some embodiments, the stationary phase is washed with PBS.

Fractions containing EVs can be collected from the SEC column, thereby purifying EVs from the liquid biological sample. For example, fractions 6-21 or fractions 12-27 can be collected, depending on the size of the column. In some embodiments, fractions 6-21 can be collected 10 mL columns. In other embodiments, fractions 12-27 can be collected for 20 mL columns. A smaller number of fractions can also be collected from the SEC column. For example, higher purity of EVs could also be achieved by taking a smaller number of the fractions (e.g., 7-9 instead of 7-10), albeit with lower yield.

While not necessary, following purification of EVs using any of the methods of the present disclosure, the purified extracellular vesicles can be further purified by any means known in the art. In addition, the methods for purification of EVs as described herein can be combined with each other, and with other EV purification methods known in the art. For example, in some embodiments, cation exchange chromatography, size exclusion chromatography, such as gel permeation columns, centrifugation or density gradient centrifugation, and filtration methods can be used in combination with the methods of the disclosure. As another example, the EV purification methods of the disclosure can be used differential centrifugation, anion exchange and/or gel permeation chromatography, sucrose density gradients, organelle electrophoresis, magnetic activated cell sorting (MACS), or with a nanomembrane ultrafiltration concentrator.

In some embodiments, the fractions collected from the SEC column are further subjected to a cation exchange chromatography resin. The cation exchange chromatography resin comprises a stationary phase comprising a functional group selected from the group consisting of sulpfhydryl, sulfonate, sulfate, carboxymethyl, sulfoethyl, sulfopropyl, phosphate and sulfonate. In some embodiments, the cation exchange chromatography resin is Fractogel® EMD-SO3-resin.

In some embodiments, the fractions collected from the SEC column are further subjected to a mixed mode resin (MMR) beads capable of capturing molecules smaller than about 700 kDa. In some embodiments, the MMR beads are Capto™ Core 700. In another embodiment, the fractions collected from the SEC column are further subjected to a cation exchange chromatography resin and MMR beads capable of capturing molecules smaller than about 700 kDa.

In some embodiments, the methods of the present application recover at least about 1.5 fold, about 2 fold, about 2.5 fold, about 3 fold, about 3.5 fold, about 4 fold, about 4.5 fold, about 5 fold, about 6 fold, about 7 fold, about 8 fold, about 9 fold, about 10 fold, or more EV from the samples, e.g., plasma and CSF samples, when compared to existing methods in the art.

Highly abundant proteins, such as albumin and immunoglobulins, may hinder purification of EVs from a biological sample. Therefore, the methods of the disclosure may be used with a system that utilizes multiple antibodies that are specific to the most abundant proteins found in blood. Such a system can remove up to several proteins at once, thus unveiling the lower abundance species such as cell-of-origin specific exosomes. Other known methods for EV purification include high abundant protein removal methods as described in Chromy et al. J. Proteome Res 2004; 3:1120-1127. In another embodiment, the purification of EVs from a biological sample may also be enhanced by removing serum proteins using glycopeptide capture as described in Zhang et al, Mol Cell Proteomics 2005; 4:144-155.

Isolation and/or Enrichment of Cell Type-Specific EVs

Extracellular vesicles isolated from a biological sample obtained from a subject can be further enriched through positive selection, negative selection, or a combination of positive and negative selection. As used herein, the term “isolating” or “enriching” refers to increasing the concentration or density of extracellular vesicles or a subpopulation of extracellular vesicles in a sample and, or, removing non-EV substances (e.g., proteins, cells) from a sample.

EVs can be further isolated and/or enriched based on the source or type of the cells. In some embodiments, cell type-specific EVs can be isolated and/or enriched based on differences in the biochemical properties of EVs. For example, cell type-specific EVs can be further isolated and/or enriched based on antigen, nucleic acid, metabolic, gene expression, or epigenetic differences. In some embodiments, based on antigen differences, antibody-conjugated magnetic or paramagnetic beads in magnetic field gradients or fluorescently labeled antibodies with flow cytometry are used. Cell type-specific EVs can also be enriched based on other biochemical properties known in the art. For example, EVs can be enriched based on pH or motility. Further, in some embodiments, more than one method is used to enrich for EVs. In other embodiments, samples are enriched for EVs using antibodies, ligands, or soluble receptors.

Because EVs often carry surface molecules such as antigens from their donor cells, surface molecules may be used to identify, isolate and/or enrich for EVs from a specific donor cell type. In some embodiments, surface markers are used to positively enrich a subpopulation of EVs from one or more cell types. In some embodiments, cell surface markers that are not found on EV populations are used to negatively enrich vesicles by depleting cell populations. Flow cytometry sorting may also be used to further enrich for EVs using cell surface markers or intracellular or extracellular markers conjugated to fluorescent labels. Intracellular and extracellular markers may include nuclear stains or antibodies against intracellular or extracellular proteins preferentially expressed in vesicles.

In some embodiments, the EVs isolated from a biological sample, e.g., a bodily fluid, e.g., CSF or plasma, are enriched for those originating from a specific tissue, for example, brain, lung, pancreas, stomach, intestine, bladder, kidney, ovary, testis, skin, colorectal, breast, prostate, brain, esophagus, liver, placenta, fetus cells.

In some embodiments, the cell type-specific EVs are isolated and/or enriched. In some embodiments, the cell type-specific EVs, e.g., brain- and/or neuron-specific EVs, are isolated and/or enriched from a sample, e.g., a biological sample such as plasma or cerebrospinal fluid, based on the biomarkers of the present invention, e.g., one or more biomarkers in Tables 1-5.

In some embodiments, EVs enriched in the biological samples are brain-specific EVs. In one embodiment, the biomarkers for brain-specific EVs include one or more biomarkers in Tables 1-5, or any combinations thereof.

In some embodiments, EVs enriched in the biological samples are neuron-specific EVs. In one embodiment, the biomarkers for neuron-specific EVs include one or more biomarkers in Tables 1 and 5, or any combinations thereof. In one embodiment, the biomarkers for neuron-specific EVs include on or more biomarker selected from the group consisting of GABRG2, SVOP, SLC32A1, GRM7, GABRB3, CHRNB2, SLC12A5, GRM8, PTPRT, SLC6A17, PCDHAC2, PLPPR4, C11orf87, SORCS3, CALY, PTPRR, KIAA1549L, HCN1, CDH18, TMEM132D, GPR158, FRRS1L, ATP2B3, GRIA4, ST8SIA3, HS6ST3, SEZ6, and NRXN3.

In some embodiments, EVs enriched in the biological samples are astrocyte-specific EVs. In one embodiment, the biomarkers for astrocyte-specific EVs include one or more biomarkers in Table 2, or any combinations thereof.

In some embodiments, EVs enriched in the biological samples are oligoodendrocyte specific EVs. In one embodiment, the biomarkers for oligodendrocyte-specific EVs include one or more biomarkers in Table 3, or any combinations thereof.

In some embodiments, EVs enriched in the biological samples are microglial-specific EVs. In one embodiment, the biomarkers for microglia-specific EVs include one or more biomarkers in Table 4, or any combinations thereof.

In some embodiments, isolation of cell type-specific EVs can be achieved through one or more purification or isolation steps. The one or more purification steps can include, but are not limited to, an immuno-isolation, a microfluidic affinity based purification, a magnetic based purification, a pull-down purification, or a fluorescence activated vesicle sorting-based purification. Commercial precipitation kits like ExoQuick™ and Total Exosome Isolation™ precipitation solutions are also available. Such kits are easy to use with only 1 or 2 steps and do not require any expensive equipment or advanced technical know-how.

In some embodiments, isolation can be performed using immuno-isolation with one or more antibody against the one or more biomarkers of the present invention, e.g., the one or more markers in Tables 1-5. Immuno-isolation can be performed using a bait/prey strategy. In some embodiments, the bait molecule can be a bait protein, such as an antibody, e.g., a monoclonal antibody directed against a prey EV biomarker of the present invention. In some embodiments, the bait molecule can also be an RNA aptamer. If several prey EVs are to be combined for purification, a mix of corresponding monoclonal antibodies directed against each of the said prey EV biomarkers to be pull-up can be used.

In some embodiments, the bait molecule is recognized by an affinity ligand. Said affinity ligand can be a divalent metal-based complex, a protein, a peptide such as fusion protein tag or more preferentially an antibody.

In some embodiments, the bait molecule or the affinity ligand is immobilized or “coupled” directly, or indirectly to a solid substrate material such as by formation of covalent chemical bonds between particular functional groups on the ligand (for example primary amines, thiols, carboxylic acids, aldehydes) and reactive groups on the substrate. A substrate, or a matrix, in the affinity purification steps of the method of the invention can be any material to which a biospecific ligand (i.e., the bait molecule or the affinity ligand) is coupled. Useful affinity supports may be those with a high surface-area to volume ratio, chemical groups that are easily modified for covalent attachment of ligands, minimal nonspecific binding properties, good flow characteristics and/or mechanical and chemical stability. Several substrates may be utilized as solid substrate, including for example agarose, cellulose, dextran, polyacrylamide, latex or controlled pore glass. Magnetic particles may also be used as a substrate instead of beaded agarose or other porous resins. Their small size provides the sufficient surface area-to-volume ratio needed for effective ligand immobilization and affinity purification. Magnetic beads may be produced as superparamagnetic iron oxide particles that may be covalently coated with silane derivatives. The coating makes the beads inert (i.e., to minimize nonspecific binding) and provides the particular chemical groups needed for attaching any affinity ligands of interest. Affinity purification with magnetic particles is generally not performed in-column. Instead, a few microliters of beads may be mixed with several hundred microliters of sample as a loose slurry. During mixing, the beads remain suspended in the sample solution, allowing affinity interactions to occur with the immobilized ligand. After sufficient time for binding has been given, the beads are collected and separated from the sample using a powerful magnet.

In some embodiments, a pull-down assay can be performed for the purification or isolation of a cell type-specific EVs by pulling-down of one or more specific EV biomarkers of the present invention, e.g. using one or more antibody against each of the one or more biomarkers in Tables 1-5. Said EV biomarkers can be specific of at least one cell type and advantageously lead to enriching in EVs from said selected cell type.

In some embodiments, the at least one or more purification steps for the purification of a cell type-specific EV subpopulation comprise a pull-down purification. In such pull-down purification, the prey EV biomarker is generally a (trans) membrane protein, which has been found to be expressed in a cell type or a cell subtype. The bait protein is preferentially a monoclonal antibody directed against any of the prey EV biomarker(s) which is to be pulled-up. Magnetic beads such as magnetic nucleic acid binding beads, or silica beads functionalized with silane (for example Dynabeads® from Thermo Fisher Scientific, such as Dynabeads® MyOne Silane Beads from Thermo Fisher Scientific) coated with an affinity ligand for the bait protein can be used to isolate said bait protein bound to said prey EV biomarker(s). The affinity ligand is preferentially a class specific or a species-specific antibody. As a matter of example, magnetic beads coated with anti-mouse antibodies can be used together with monoclonal mouse antibodies directed against a specific surface protein of a cell type or cell subtype subpopulation of EV. Generally, a control antibody, such as a mouse mCherry monoclonal antibody, can be used.

A pull-down assay can therefore be used to illustrate and validate the purification, or isolation of one or more EV subpopulations each expressing at least one specific membrane protein marker. The purification or isolation of EV subpopulations by at least one specific prey EV biomarker can be further confirmed using western blot or qRT-PCR.

### E. Methods For Diagnosis and Prognosis of Diseases

The present invention also provides methods for diagnosing or prognosing a disease, e.g., a neurodegenerative disorder, in a subject, identifying a subject at risk of a disorder, or prescribing a therapeutic regimen or predicting benefit from therapy in a subject having a disorder.

Once the cell type-specific and/or organ-specific EVs, e.g., the brain- and/or neuron-derived EVs, are isolated and/or enriched based on the presence of the one or more biomarkers of the present invention, profiling the molecular contents of these cell type-specific and/or organ-specific EVs provides great insight for both early detection and better understanding of the pathology. For example, since human brain and/or neurons are generally not accessible to biopsy, reading out the molecular contents of brain and/or neurons through the isolation of brain- and/or neuron-derived EVs from accessible biofluids could provide a unique view through which to understand the brain pathology.

Accordingly, in one aspect, the present invention provides a method for diagnosing, prognosing, or identifying a subject at risk of developing a neurodegenerative disorder in a subject comprising: (a) obtaining a biological sample from the subject; (b) isolating brain-specific extracellular vesicles from the biological sample based on the presence of a biomarker in the isolated extracellular vesicles, wherein the biomarker comprises one or more biomarkers selected from Tables 1-5; (c) extracting protein and/or RNA from the isolated brain-specific extracellular vesicles; and (d) analyzing the extracted protein and/or RNA from the isolated brain-specific extracellular vesicles, thereby diagnosing, prognosing, or identifying the subject at risk of developing the neurodegenerative disorder.

In some embodiments, the biological sample comprises a liquid biological sample. In some embodiments, the liquid biological sample is selected from the group consisting of whole blood, serum, plasma, cerebrospinal fluid, spinal fluid, amniotic fluid, aqueous humor, vitreous humor, bile, breast milk, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof.

In some embodiments, the extracellular vesicles are neuron-specific, astrocyte-specific, oligodendrocyte-specific, and/or microglial-specific.

In some embodiments, the extracellular vesicles are neuron-specific and wherein the one or more biomarkers are selected from Tables 1 and 5. In some embodiments, the one or more biomarkers are selected from the group consisting of GABRG2, SVOP, SLC32A1, GRM7, GABRB3, CHRNB2, SLC12A5, GRM8, PTPRT, SLC6A17, PCDHAC2, PLPPR4, C11orf87, SORCS3, CALY, PTPRR, KIAA1549L, HCN1, CDH18, TMEM132D, GPR158, FRRS1L, ATP2B3, GRIA4, ST8SIA3, HS6ST3, SEZ6, and NRXN3.

In some embodiments, the extracellular vesicles are astrocyte-specific and wherein the one or more biomarkers are selected from Table 2.

In some embodiments, the extracellular vesicles are oligodendrocyte-specific and wherein the one or more biomarkers are selected from Tables 3.

In some embodiments, the extracellular vesicles are microglial-specific and wherein the one or more biomarkers are selected from Table 4.

The protein and/or RNA content of EVs has been found to correlate to the protein and/or RNA content of the corresponding cell. Therefore, analyzing the protein and/or RNA content of the EVs provides both qualitative and quantitative information about the cellular RNA content of the corresponding cells. Advantageously, this makes it possible to provide non-invasive diagnostic methods. Indeed, the analysis (whether by DNA/RNA sequence, transcriptome profiling, qRT-PCR, microarray, proteome profiling, or mass spectrometry, etc) is performed on a biological sample derived from body fluids, such as derived from blood or cerebrospinal fluid. Such fluids are more easily and readily available than the corresponding organs, e.g., brain. Correspondingly, the present invention provides diagnostic methods that are non-invasive and yet reliable.

In some embodiments, the protein and/or nucleic acid content of the cell type-specific and/or organ-specific EVs is extracted and analyzed. In some embodiments, the extracted nucleic acid comprises messenger RNA (mRNA), microRNA (miRNA), long non-coding RNA (lncRNA), small non-coding RNA, DNA, and any other full length or fragment of RNAs or DNAs. Protein and/or nucleic acid extraction can be performed using any known methods or commercial kits in the art.

Analyses of the RNA content in EVs can be performed using any transcriptomics method, such as RNA sequencing, DNA sequencing, reverse transcription polymerase chain reaction (RT-PCR), or quantitative reverse transcription polymerase chain reaction (qRT-PCR), or array analysis. In some embodiments, analyzing the extracted nucleic acids from the isolated brain-specific extracellular vesicles comprises genome-wide analysis, or transcriptome profiling.

In other embodiments, analyzing the extracted nucleic acids from the isolated brain-specific extracellular vesicles comprises analyzing a gene of interest, wherein the gene of interest is associated with the neurodegenerative disorder. For example, the presence or absence of saithed gene of interest, analyzing for one or more allelic variants or mutations of the gene of interest, testing for presence or absence of the allelic variants or mutations are tested.

Similarly, analysis of the protein content in EVs can be performed using any proteomic methods known in the art, such as proteomic profiling, mass spectrometry, immunoassay, ELISA, fluorescence activated cell sorting (FACS), SDS-polyacrylamide gel electrophoresis (SDS-PAGE), or Western blot analysis.

In some embodiments, analyzing the extracted protein from the isolated brain-specific extracellular vesicles comprises analyzing a protein of interest, wherein the protein of interest is associated with the neurodegenerative disorder. In some embodiments, analyzing the extracted protein comprises testing for the presence or absence of the protein of interest, analyzing for one or more mutations in the protein of interest, e.g., a deletion, an addition, a substitution, a truncation, or a modification, e.g., a protein with an altered state of post-translational modification, or an epigenetic change for the protein of interest, and testing for presence or absence of the mutations or modifications.

The neurodegenerative disorder for diagnosis or prognosis by the methods of the present invention include, but are not limited to, Alzheimer's disease (AD), Huntington's Disease, multiple sclerosis, vascular disease dementia, frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease.

Genes/proteins of interest that are associated with a neurodegenerative disorder are known in the art. Examples of genes/proteins associated with Parkinson's disease include but are not limited to a-synuclein, DJ-1, LRRK2, PINK1, Parkin, UCHL1, Synphilin-1, and NURR1. Examples of Alzheimer's disease associated genes/proteins may include, but are not limited to, Tau and associated post-translational modifications (p-Tau), the very low density lipoprotein receptor protein (VLDLR) encoded by the VLDLR gene, the ubiquitin-like modifier activating enzyme 1 (UBA1) encoded by the UBA1 gene, or the NEDD8-activating enzyme El catalytic subunit protein (UBE1C) encoded by the UBA3 gene. Examples of genes/proteins associated with amyotrophic lateral sclerosis may include SOD1 (superoxide dismutase 1), ALS2 (amyotrophic lateral sclerosis 2), FUS (fused in sarcoma), TARDBP (TAR DNA binding protein), VAGFA (vascular endothelial growth factor A), VAGFB (vascular endothelial growth factor B), and VAGFC (vascular endothelial growth factor C), and any combination thereof. Analyzing these genes or proteins of interest in brain- and/or neuron-derived EVs provides an insight in the health of the brain for the subject.

In some embodiments, analysis of the cell type-specific and/or organ-specific extracellular vesicles, e.g., brain- and/or neuron-specific EVs, may be made over a particular time course in various intervals to assess a subject's progression and pathology. For example, analysis may be performed at regular intervals such as one day, two days, three days, one week, two weeks, one month, two months, three months, six months, or one year, in order to track the level and characterization of brain- and/or neuron-derived EVs as a function of time. In the case of existing patients, this provides a useful indication of the progression of the disease and assists medical practitioners in making appropriate therapeutic choices based on the increase, decrease, or lack of change in the content of the brain- and/or neuron-derived EVs. For those at risk of neurodegenerative diseases, the protein and/or RNA content of the brain- and/or neuron-derived EVs may provide an early warning or diagnosis.

### F. KITS

Another aspect of the invention encompasses kits for isolating a cell type-specific and/or organ-specific extracellular vesicle, e.g., a brain- and/or neuron-specific EV, from a biological sample obtained from a subject; and kits for detecting a neurodegenerative disorder in a subject.

Kits for isolating brain-specific extracellular vesicles may include one or more of the following: (a) one or more reagents for detecting the presence of a biomarker on the surface of the extracellular vesicle, wherein the biomarker comprises one or more biomarkers selected from Tables 1-5; (b) means for isolating the brain-specific extracellular vesicles based on the presence of the biomarker; and (c) a set of instructions for detecting the presence of the biomarker, and/or isolating the brain-specific extracellular vesicles. In some embodiments, the one or more reagents for detecting the presence of the biomarker on the extracellular vesicles is an antibody or an aptamer that binds the biomarker. In some embodiments, the kits further comprise means for isolating a biological sample from the subject.

Kits for detecting a neurodegenerative disorder may include one or more of the following: (a) one or more reagents for detecting the presence of a biomarker on the surface of the extracellular vesicle, wherein the biomarker comprises one or more biomarkers selected from Tables 1-5; (b) means for isolating the brain-specific extracellular vesicles based on the presence of the biomarker; (c) one or more reagents for measuring the level of a gene associated with the neurodegenerative disorder in the isolated brain-specific extracellular vesicles; and (d) a set of instructions for detecting the presence of the biomarker, isolating the brain-specific extracellular vesicles, and/or measuring the level of the gene associated with the neurodegenerative disorder. In some embodiments, the kits further comprise means for isolating a biological sample from the subject.

A variety of kits having different components are contemplated by the current invention. Generally, the kit will include means for collecting a biological sample or extracellular vesicles, means for detecting one or more biomarkers in the extracellular vesicles, and instructions for use of the kit contents. In certain embodiments, the kit comprises a means for enriching or isolating a subpopulation of extracellular vesicles, e.g., cell type-specific and/or organ-specific extracellular vesicles, in a biological sample. In further embodiments, the means for enriching or isolating extracellular vesicles comprises reagents necessary to enrich or isolate extracellular vesicles from a biological sample. In some embodiments, the one or more reagents for detecting the presence of a biomarker on the surface of the extracellular comprises an antibody for the biomarker. In certain embodiments, the kit comprises a means for detecting and/or quantifying the level of a gene of interest associated with a neurodegenerative disorder in the cell type-specific and/or organ-specific extracellular vesicles. In further embodiments, the means for quantifying the amount of a gene of interest comprises reagents necessary to detect the amount of a gene of interest.

The contents of all documents cited or referenced herein and all documents cited or referenced in the herein cited documents, together with any manufacturer's instructions, descriptions, product specifications, and product sheets for any products mentioned herein or in any document incorporated by reference herein, GenBank Accession and Gene numbers, and published patents and patent applications, are hereby incorporated by reference, and may be employed in the practice of the invention. Those skilled in the art will recognize that the invention may be practiced with variations on the disclosed structures, materials, compositions and methods, and such variations are regarded as within the ambit of the invention.

This invention is further illustrated by the following examples which should not be construed as limiting.

### EXAMPLES

### Examples 1: Identification of Markers for Isolating Neuron-Specific Extracellular Vesicles

### Selection of Neuron-Specific EV Marker Candidates Based on Gene Expression

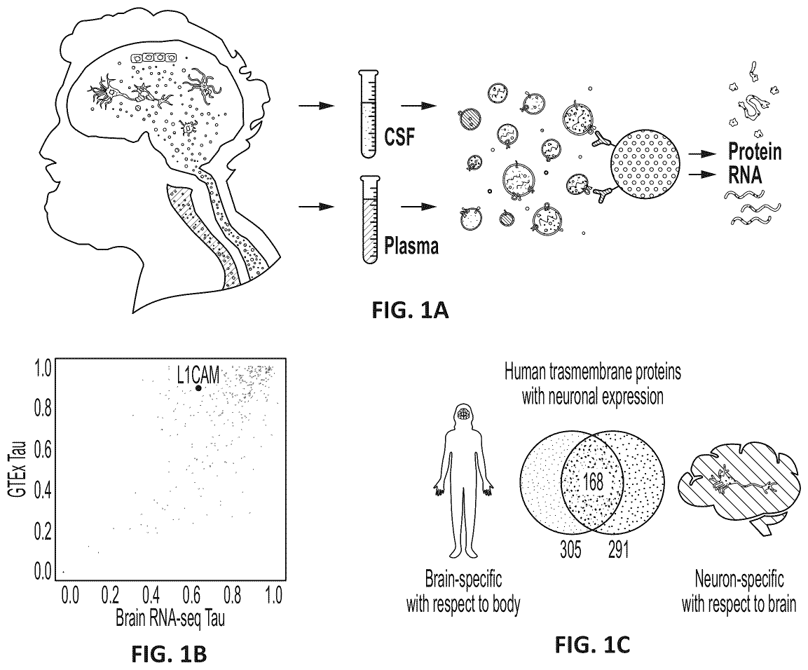

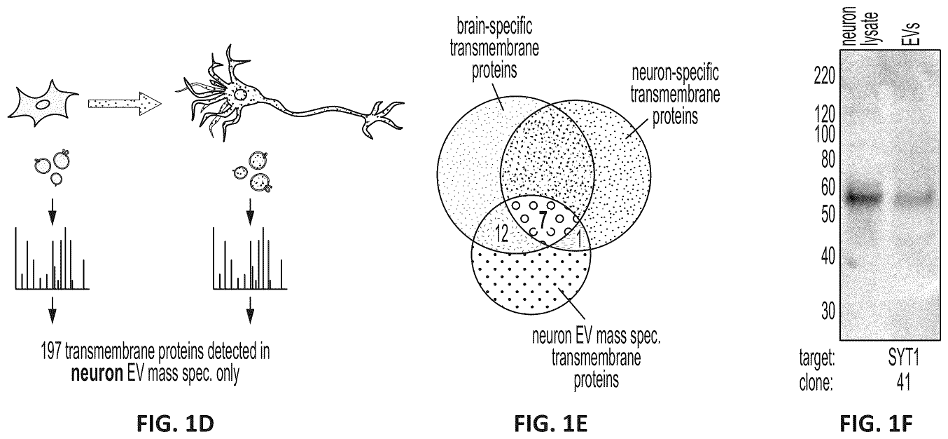

All human proteins were assessed for their potential as handles for neuron-specific EV isolation from human biofluids such as CSF and plasma (FIG. 1A). The goal was not to definitively decide which markers would work best, but rather to construct a simple framework for determining which markers should be prioritized for experimental validation. The first requirement is that a potential marker must have an annotated transmembrane domain, as this is necessary for it to be used in immuno-isolation of EVs. Out of the 20,375 reviewed proteins in the protein database UniProt, 4,845 have annotated transmembrane domains (33).

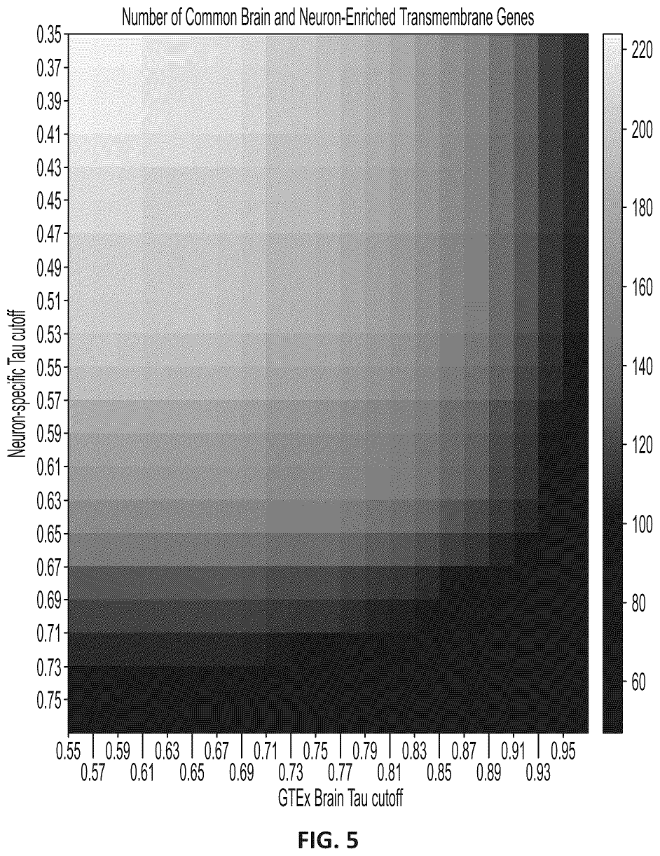

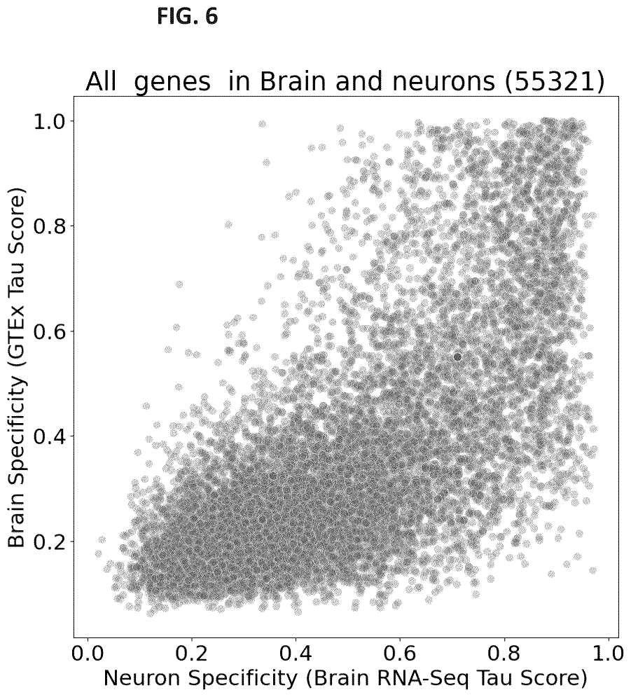

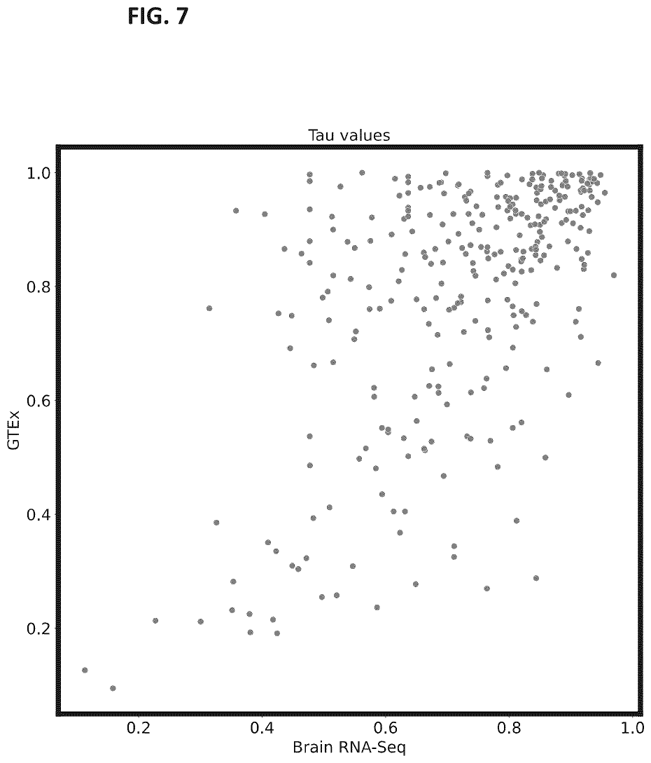

The second requirement is cell-type specific gene expression of the marker in the desired cell and tissue of origin. Thus, candidate markers should be highly enriched in neurons relative to other cell types of the brain and highly enriched in the brain relative to other organs within the human body. To evaluate cell type-specific gene expression, RNA-Seq data of the major cell types isolated from human brain tissue via immuno-panning were analyzed (34). To evaluate organ-level expression, the GTEx RNA-Seq dataset of human organs were analyzed (35). The gene expression specificity index, Tau, was used to calculate specificity for each gene (36) as it has been shown to be particularly robust relative to other methods (37). Selecting a Tau cutoff of 0.7 (FIG. 1B, FIGS. 5-7), it was found that there are 291 genes encoding transmembrane proteins specifically expressed in neurons relative to other cell types of the brain and 305 genes encoding transmembrane proteins specific to the brain relative to other organs. Overlapping these two lists, 168 transmembrane proteins were found (FIG. 1C). Since some of these proteins may be expressed in neurons but not present on EVs in the biofluid of interest, it was reasoned that transmembrane proteins that exhibit cell and tissue specificity can be overlapped with proteomics datasets of EVs in the biofluid of interest.

Differentiation of Human iPS Cells to iNGN Neurons to Isolate EVs from Conditioned Media

Before investigating neuron EVs in human biofluids, it was first sought to develop a positive control (i.e., a source of pure neuron EVs). A human induced pluripotent stem (iPS) cell line with a doxycycline-inducible expression of the transcription factors Neurogenin1/2 was previously established for the rapid and highly efficient differentiation of iPS cells into neurons (38). The protocols to differentiate induced Neurogenin (iNGN) cells were scaled up and EVs were collected from their conditioned media (39). Mass spectrometry-based proteomic analysis of EVs isolated from neurons was performed and the un-induced parent iPS line was used as a control. 197 transmembrane proteins were found to be expressed on EVs from neurons but not iPS cells (FIG. 1D). Of these 197 proteins, seven pass the cell and tissue specificity threshold (FIG. 1E). Using Western blotting, it was confirmed the presence of one of these markers, SYT1 in both neuron cell lysate and neuron EVs (FIG. 1F).

Development of EV Immuno-Isolation Protocol Using EVs from Cell Culture

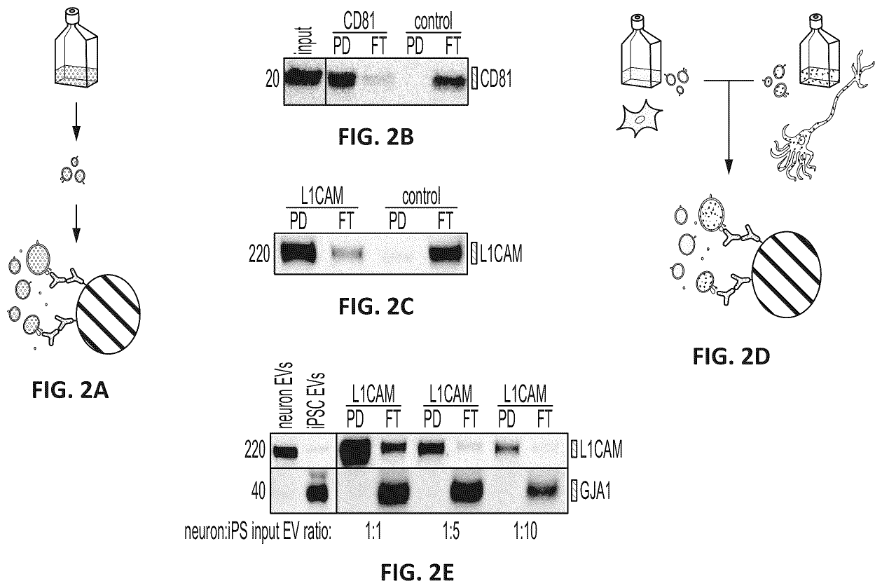

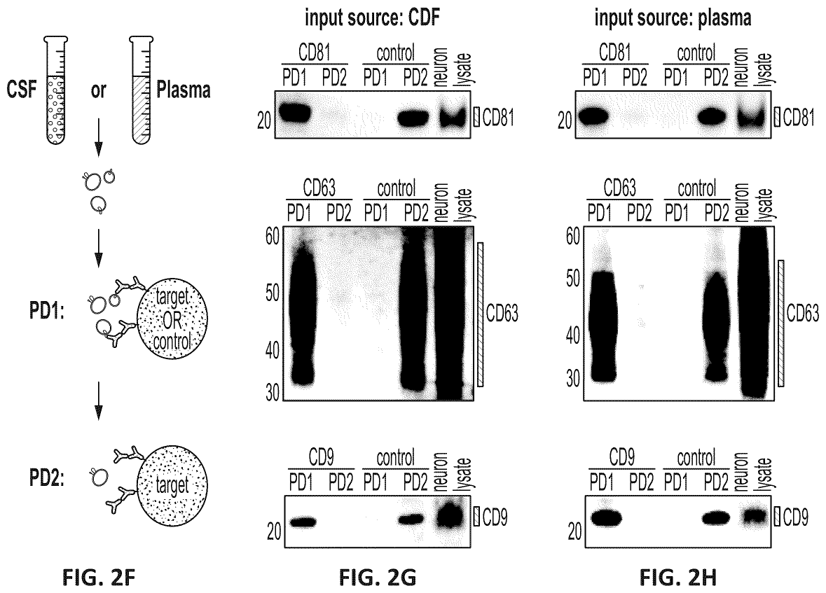

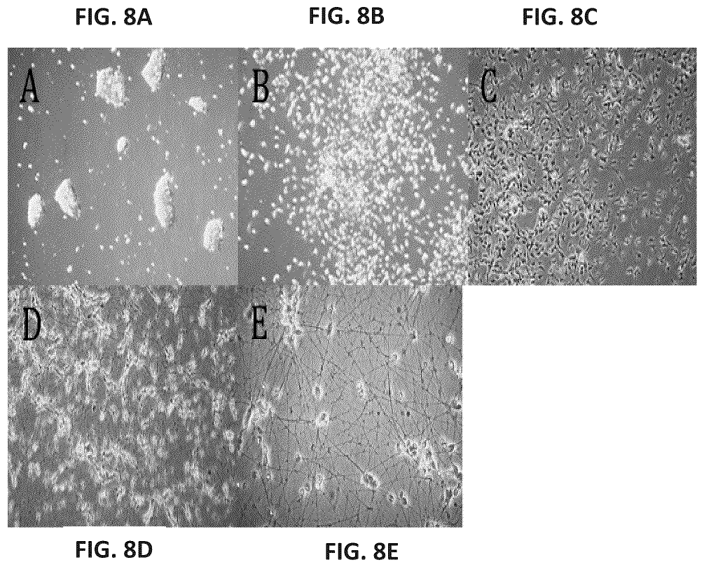

An immuno-isolation protocol was developed to capture subsets of EVs displaying a specific marker. Since it was expected that EVs from neurons were a small subset of total EVs in a biofluid such as plasma, the protocol needs to be highly efficient and specific. To simplify this goal, the human K562 cell line was selected (FIG. 2A). The immuno-isolation was first optimized using the widely expressed tetraspanin CD81. After purifying EVs from the conditioned media of K562 cells using differential ultracentrifugation (39), immuno-isolation was performed and the efficiency was evaluated via Western blotting of CD81 in the pulldown vs. flow-through fraction. To ensure that the immuno-isolation is specific for the target protein, CD81 was also measured when EVs were immuno-isolated using the same procedure but with a non-specific antibody. Using this system, several parameters such as antibody conjugation strategy, bead and antibody quantity and ratio, etc. were systematically optimized. The final protocol led to immuno-isolation with high efficiency and specificity (FIG. 2B, FIGS. 8A-8E).

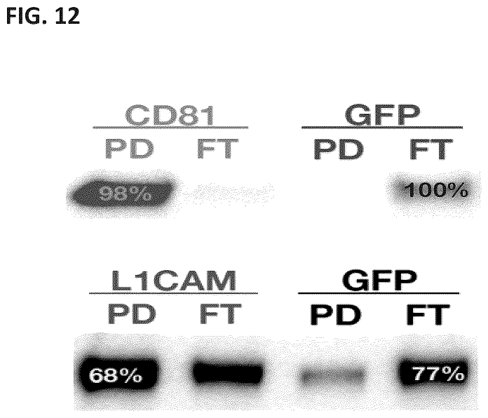

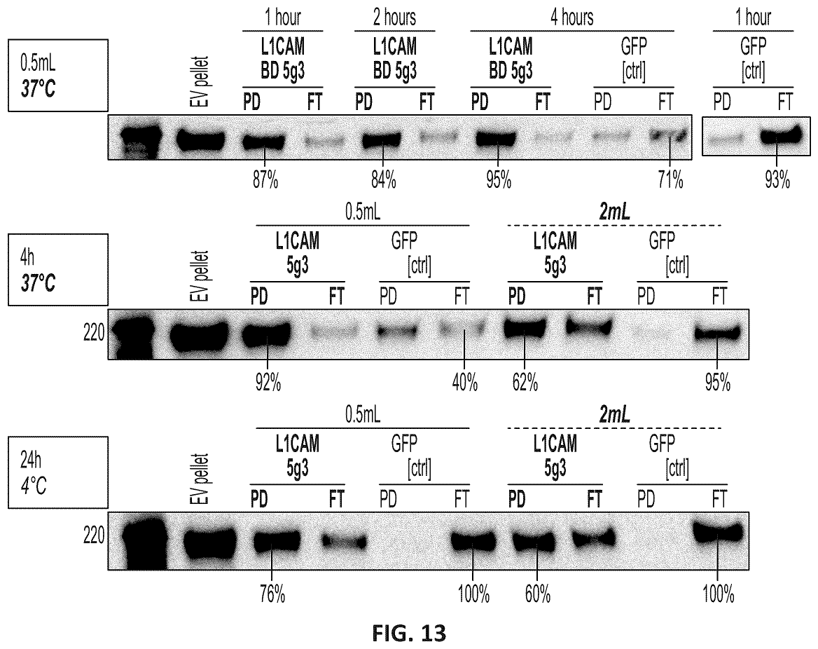

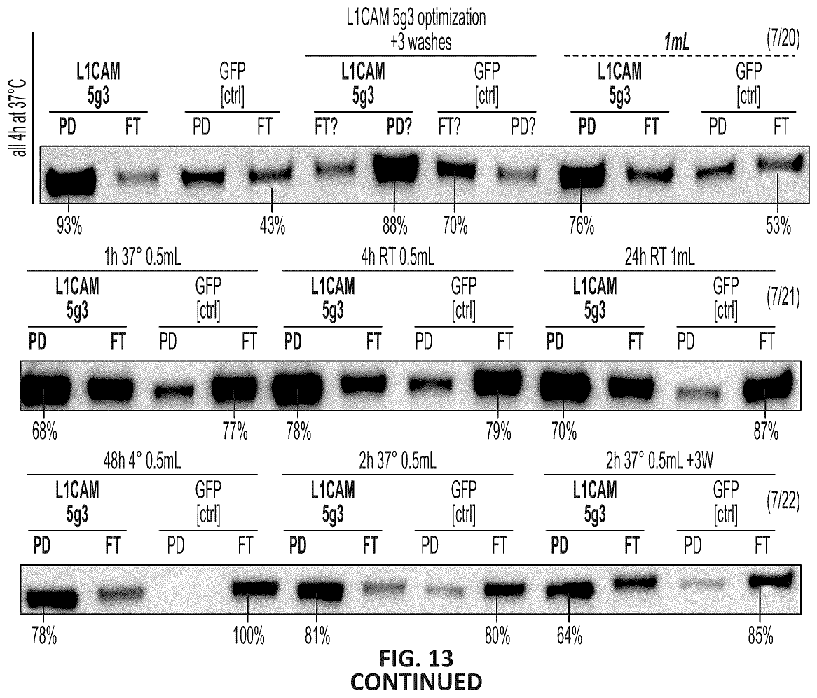

Next, the optimized general cell culture EV immuno-isolation protocol was applied to EVs from iNGN neurons. The proteomic data detected the presence of L1CAM and NCAM1 on neuron EVs. Although they did not pass the Tau specificity score, these two proteins, and L1CAM in particular, have been used by others as targets for immuno-isolation in plasma. Although it was ultimately showed that L1CAM is not a viable marker in human CSF and plasma (32), before analyzing L1CAM in biofluids, L1CAM was analyzed on iNGN EVs as a testbed for neuron EV immuno-isolation. The previously used antibodies were directly compared and further optimizations were performed (FIGS. 12 and 13) to arrive at a highly efficient and specific L1CAM pulldown in iNGN EVs (FIG. 2C). In order to model cell-type specific EV capture from biofluids, L1CAM-positive EVs from neurons were mixed with L1CAM-negative EVs from undifferentiated iPS cells (FIG. 2D). GJA1, a protein present on iPS EVs but not neuron EVs, was monitored after mixing EVs from both cell types and it was confirmed that GJA1 was not detected on beads with L1CAM antibodies (FIG. 2E). These experiments confirmed that the immuno-isolation is specific, even with a low proportion of neuron EVs relative to non-neuron EVs.

Immuno-Isolation of EV Subsets from Human CSF and Plasma

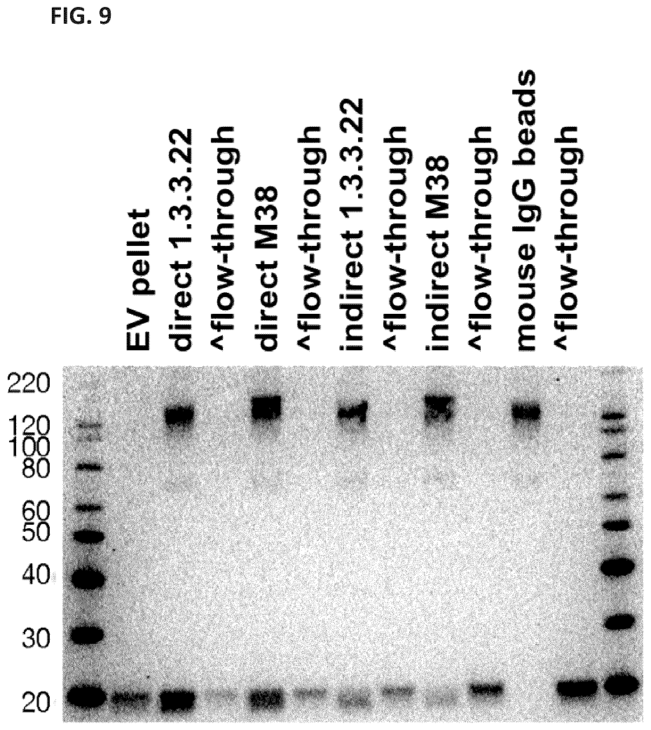

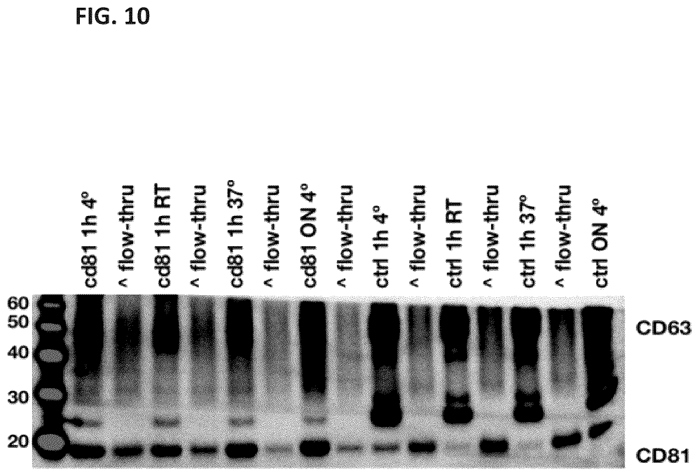

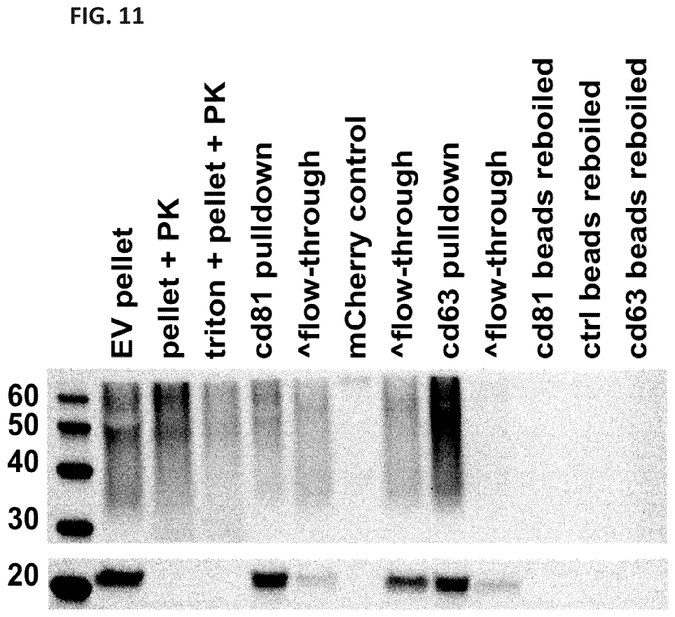

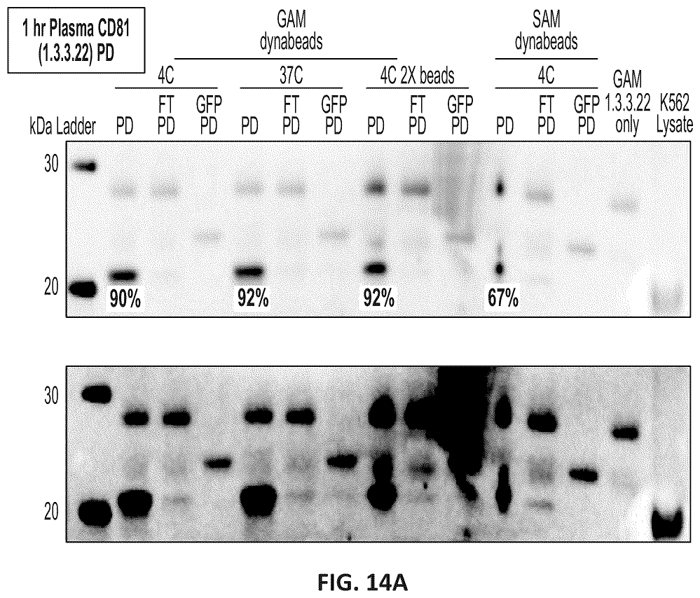

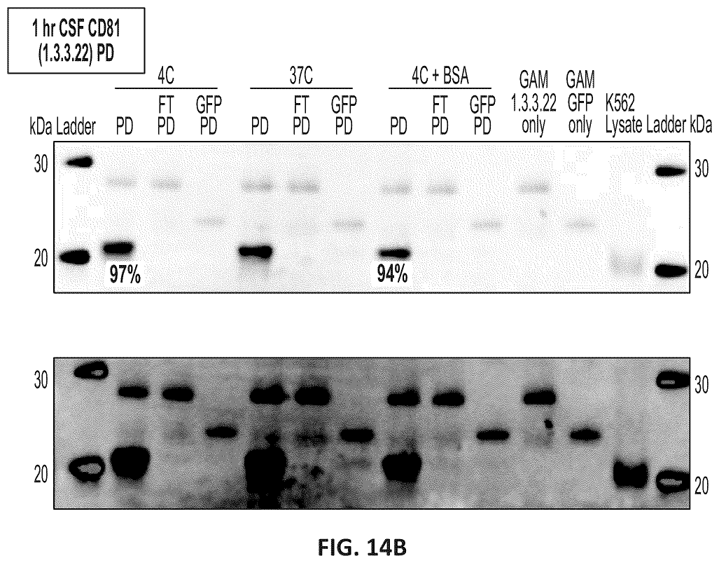

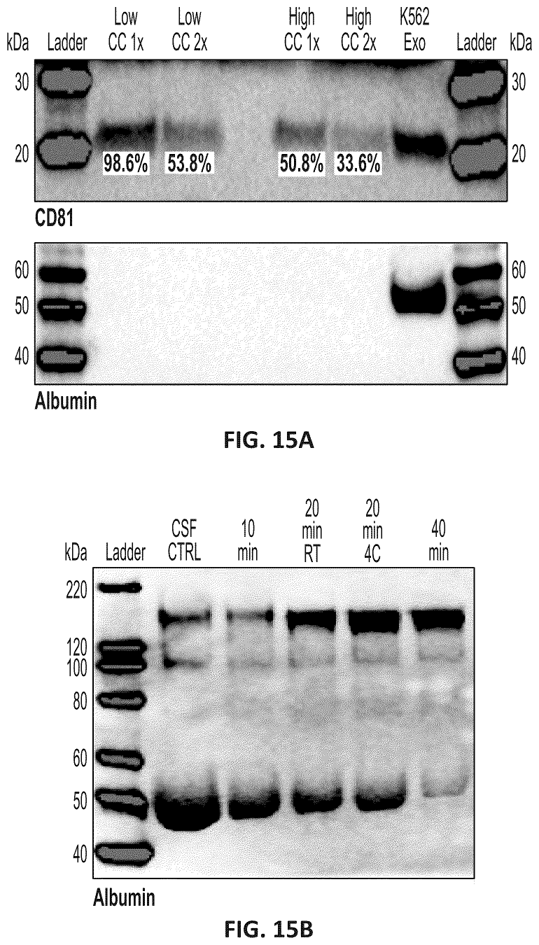

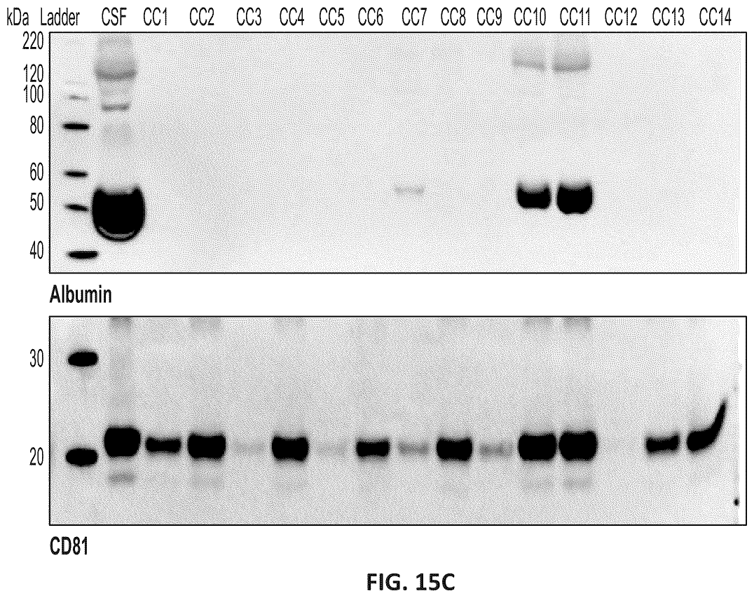

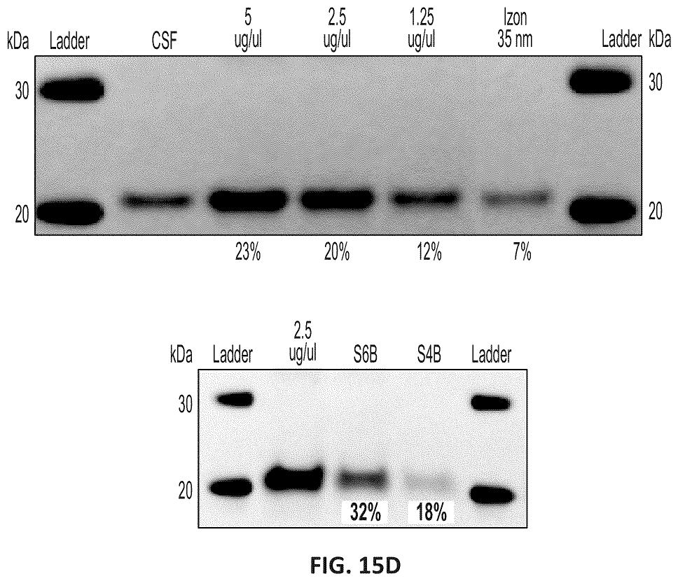

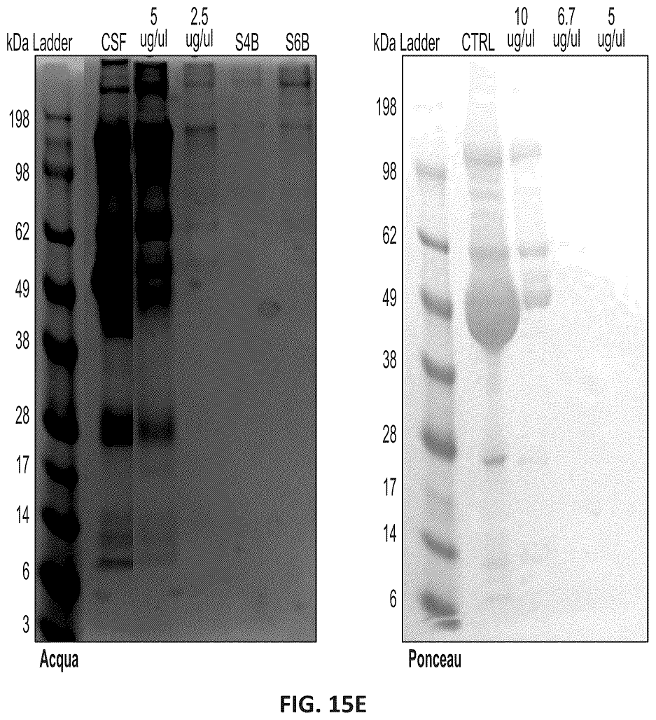

After developing the immuno-isolation methods in EVs from cell culture, the methods were tested in human biofluids (FIG. 2F). With some optimizations (FIGS. 9-11, FIGS. 14A-B. and FIGS. 15A-E), EVs containing the tetraspanins CD9, CD63, and CD81 were immuno-isolated from human CSF (FIG. 2G) and plasma (FIG. 2H). Since the flow-through contained protein levels that were too high to run on a protein gel, a second immuno-isolation against each tetraspanin was performed on the flow-through from the first immuno-isolation (using beads with either target antibody or control antibody). Comparing the results of the first immuno-isolation to that of the second immuno-isolation suggested that the protocol is highly efficient and specific in both CSF and plasma. Thus, although contingent on having sufficiently good antibodies, this protocol was able to perform high specificity immuno-isolation from human biofluids without first purifying EVs.

### Development of High-Purity EV Isolation Method

Next, proteomics were used to assess which neuron EV markers are present in human CSF and plasma. Since free proteins (such as albumin) are many orders of magnitude more abundant than EV proteins in biofluids, however, contaminating free protein in EV preparations limits the utility of mass spectrometry in detecting EV proteins (40). Previously extensive comparisons of different EV isolation methods from CSF and plasma were done (41). Although size exclusion chromatography (SEC) was optimized to remove free proteins such as albumin by several orders of magnitude, the EV fractions in SEC still carry substantial amounts of albumin. Thus, new methods that would allow for purification of EVs from human biofluids with extraordinarily high purity while still maintaining high yield were developed. After extensive exploration, a mixed mode resin (MMR) called Capto Core 700 was identified. This resin comprises beads with an inert outer shell and pores that exclude molecules larger than 700 kDa. MMR beads have a core that contains octylamine ligands that are both hydrophobic and positively charged, efficiently trapping proteins that enter the beads. This resin was developed for use in chromatography columns (42) for viral purification, but a report has demonstrated the use of the resin for viral purification “in slurry” without the use of columns for non-enveloped, infectious virus purified from cells (43).

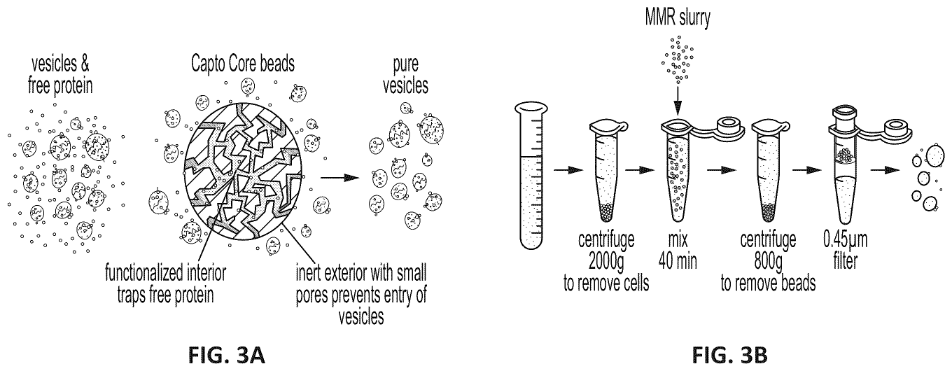

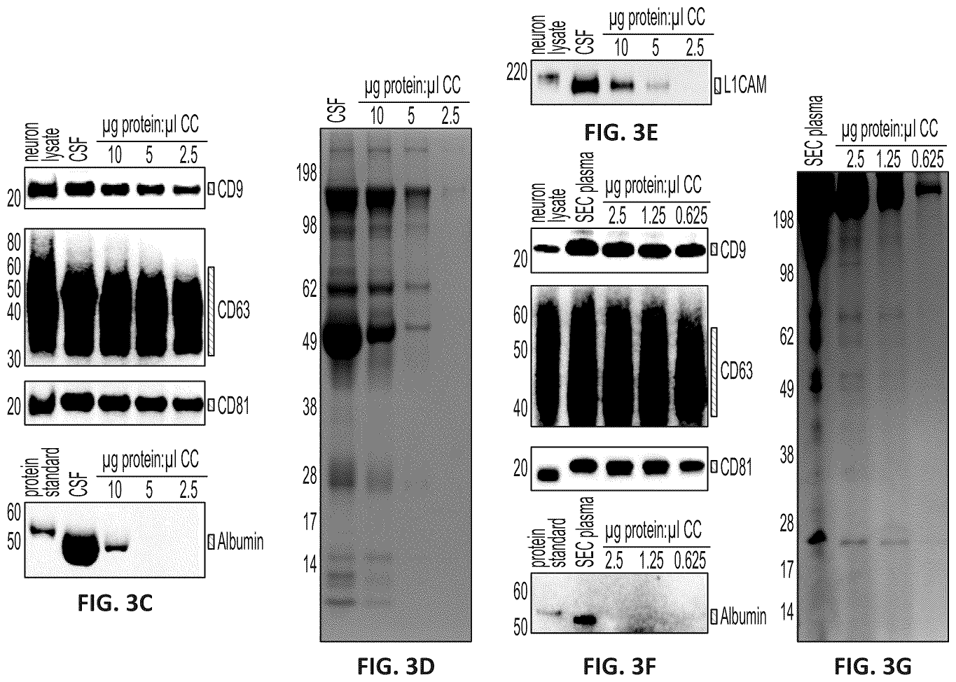

A simple method mixing MMR was developed with biofluids to purify EVs away from free proteins. Since the MMR beads bind and trap free proteins, it was reasoned that the beads could be separated after incubation with the biofluid, leaving the pure EVs (FIG. 3A). By measuring the levels of the tetraspanins CD9, CD63, and CD81 (as a proxy for EV yield) and albumin to measure free protein contamination, after various optimizations, it was able to converge on the most important parameters for EV isolation (FIG. 3B). The most important parameter was discovered to be the ratio of resin relative to total protein in the sample. An optimized method, which we called MMR Slurry, was developed and applied to CSF. It was found that by precisely increasing the ratio of resin volume to total protein in the sample, the EV purity could be “tuned”. In this way, albumin can be completely depleted (as measured by Western blot) while keeping most of the EVs (FIG. 3C, D). In agreement with the previous results (32), L1CAM also completely disappeared by Western blot upon increasing MMR amounts (FIG. 3E).

After optimizing isolation for CSF, the protocol was applied to plasma. Plasma has two orders of magnitude more protein than CSF, so the MMR Slurry method was applied to plasma after an initial purification of plasma using SEC. By applying the MMR Slurry method after SEC to 1 mL of plasma, albumin was able to be depleted to almost undetectable levels (FIG. 3F, G). Thus, the MMR Slurry method could be applied to CSF as a simple one-step purification or to plasma as a two-step protocol after SEC.

Mass Spectrometry of EVs from Human Plasma and CSF Using MMR Slurry

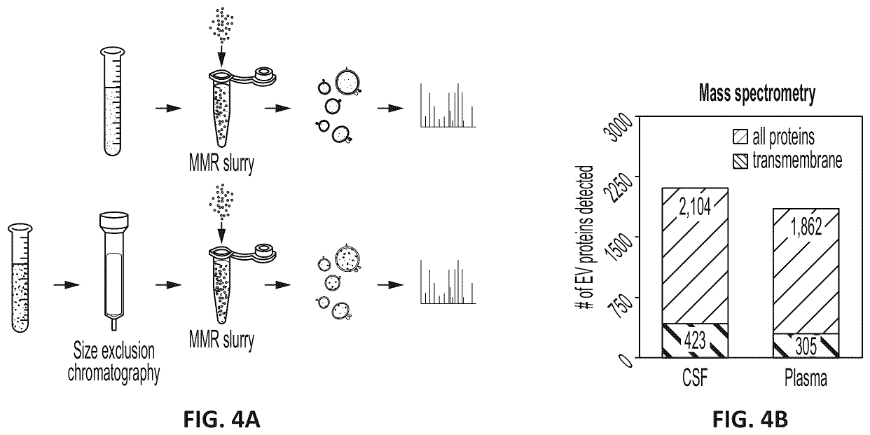

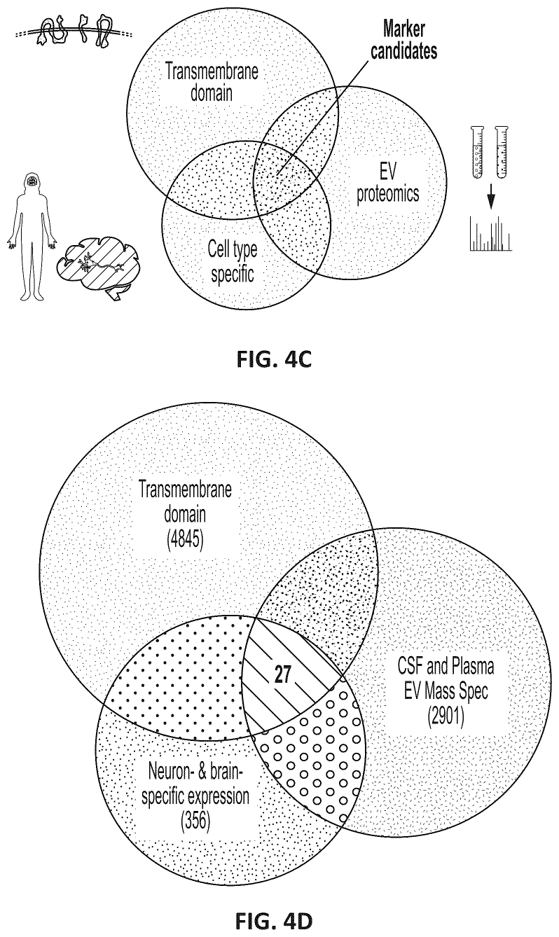

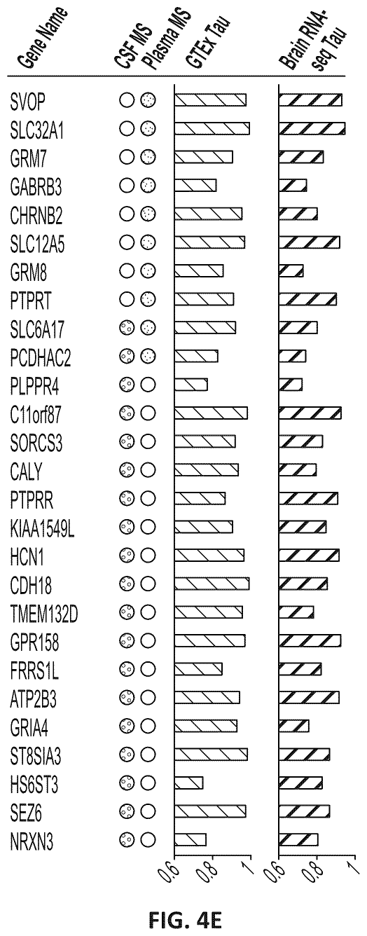

The MMR Slurry was applied to establish the EV proteome for CSF and plasma. The protocol was further optimized to increase the number of transmembrane proteins detected, and combined this high purity isolation with methods previously developed for low-input proteomics. Using the one-step MMR Slurry method for CSF or the two-step SEC and MMR Slurry method for plasma, high-quality proteomes from 1 mL of biofluid were obtained (FIG. 4A). These proteomes were comparable or deeper (in terms of the number of proteins detected) than previous studies that used much larger volumes of CSF and plasma. One limitation of using the two step SEC followed by MMR Slurry is that since lipoproteins are larger than 700 kDa, they are not removed from plasma. However, it was found that ApoB100 levels were reduced by replacing SEC with dual mode chromatography (DMC), which combines SEC with cation exchange resin (44). Isolating EVs from plasma using two-step DMC+MMR Slurry detected even more proteins than SEC+MMR Slurry (FIG. SI). The various mass spectrometry runs were combined used on 1 mL pooled human plasma or CSF to generate reference EV proteomes yielding 2104 proteins for CSF and 1862 proteins for plasma.

### Pipeline for Identification of Neuron-Specific EV Markers

Having established high-quality reference EV proteomes for CSF and plasma, these data were plugged into the computational pipeline. The list of candidate transmembrane proteins that met gene expression cutoffs was intersected with the MMR Slurry proteomics data (FIG. 4A). Overlapping these data sets, 27 candidate markers were found to meet all established criteria: transmembrane, specific cell-type and tissue expression, and found in either the EV CSF or plasma data generated (FIG. 4B). Of these 27 markers, two were detected in both CSF and plasma, with rest being present in one of the two biofluids (FIG. 4C). To further rank these candidates, existing datasets of proteins found in CSF (45, 46) and plasma (47) or EVs isolated from these biofluids (47-51) were analyzed (Tables 1-5). This comprehensive analysis allows to identify candidate markers for neuron-specific EV isolation from human biofluids.

### Discussion

In this work, a systematic and unbiased framework was provided for the identification of markers for neuron-specific EVs based on gene expression and EV proteomics data. The expression levels of all human transmembrane proteins were analyzed for enrichment in neurons relative to other cell types of the brain and in the brain relative to other organs. The markers were further prioritized for experimental validation based on proteomic data to ensure that the markers of interest are found on EVs. Towards this end, reference human CSF and plasma EV proteomics data sets that are much more extensive than those in previous studies were generated. Additionally, EVs from the conditioned media of human iNGN neurons were established as a “positive control” for endogenous neuron EVs in human biofluids and profiled their EV proteome.

In addition to generating new reference proteomics EV data sets and building a computational marker prediction pipeline, several technological advances were also made for the use of EVs as biomarkers. First, a highly optimized EV immuno-isolation protocol was developed that works in both plasma and CSF without prior EV isolation, as is generally done in other studies. Second, a novel MMR Slurry method was developed that allows for isolation of EVs with unprecedented purity, which is particularly important for proteomic profiling of EVs. The method technique can purify EVs from clinically relevant volumes of CSF by simply incubating the resin with the biofluid. The MMR Slurry was also applied to purifying high purity EVs from plasma after SEC or DMC (to remove lipoproteins). As Capto Core resin has recently been shown to remove dye or small amounts of protein when used in 96-well filter plates (52), MMR Slurry was envisioned being adaptable to a high-throughput format. This would make MMR Slurry, together with the ease and low cost of the method, particularly well-suited for EV diagnostics.

The present invention provides an important guidance for the isolation of neuron specific EVs from biofluids and the vast potential of cell-type specific EVs as a non-invasive readout of cell state in health and disease. The resources and framework introduced in this invention should be broadly applicable to identifying cell type specific EV markers for other cell types in the brain and in other organs. Profiling the molecular cargo of neuron-derived EVs presents exciting opportunities for both early detection and better understanding of brain pathology. Since human neurons are generally not accessible to biopsy, reading out the molecular contents of neurons through the isolation of neuron-derived EVs from accessible biofluids could provide an unprecedented view through which to understand the brain and what goes awry in disease.

### Materials and Methods

### Computational Marker Pipeline

Cell-type specific Tau was calculated using Brain RNA-Seq expression data (log-scaled) of the five main cell types of the brain: neuron, astrocytes, oligodendrocytes, microglia, and endothelial cells. For cell type-specific expression determination, genes with maximum expression within that cell type (neurons) relative to the other four cell types were selected if their cell-type specific Tau score was 0.7 or above. Organ-specific Tau was calculated using GTEx organ-specific RNA sequencing data. Since GTEx data contains several regions or tissues for each organ, all regions or tissues for a specific organ were averaged, resulting in one organ-level measurement. For determination of organ-level Tau, pituitary gland, tibial nerve, and testis were removed. For organ-specific expression determination, genes with maximum expression within that organ (brain) relative to the other organs were selected if their cell-type specific Tau score was 0.7 or above. Uniprot accession IDs for all human proteins were filtered for those annotated to contain a transmembrane domain. The list of candidate EV cell-type specific markers for neurons was determined by determining which genes had a Tau score of 0.7 for both neuron-specific expression in Brain RNA-Seq and brain-specific expression in GTEx, and then filtering those genes to select only those which produce transmembrane proteins. The presence of these candidates was then assessed in proteomics datasets. Python scripts for the computational marker pipeline are available on Github.

Cell Culture and EV Isolation from Cell Culture Media

K562 cells (from ATCC) were grown in Gibco IMDM with Glutamax (Thermo Fisher Scientific) supplemented with Gibco Heat-Inactivated Fetal Bovine Serum (Thermo Fisher Scientific) and Gibco Penicillin Streptomycin (Thermo Fisher Scientific). For EV isolations, cells were switched to EV-depleted media (obtained by ultracentrifugation of media for 16 hours at 120,000×g and subsequent filtration through Corning 0.22 μm filter). Previously described iNGN cells were grown in mTeSR1 media (STEMCELL Technologies) on Matrigel (Corning) coated plates. Doxycycline (Sigma Aldrich) was diluted in PBS and added to mTeSR1 at a final concentration of 0.5 g/mL to initiate differentiation. On Day 4 after Dox addition, media was switched to Gibco DMEM with Glutamax (Thermo Fisher Scientific) supplemented with B27 Serum-Free Supplement (Thermo Fisher Scientific) and Gibco Penicillin Streptomycin (Thermo Fisher Scientific). EVs from neurons were collected on Day 6 or Day 7 after Dox addition. EV isolation from cell culture was performed by differential ultracentrifugation as described in detail (REF). Cell culture media (240 mL per isolation) was centrifuged at 300×g for 10 minutes and supernatant was centrifuged again at 2000×g for 10 minutes. Supernatant was centrifuged at 16,500×g at 4C for 20 minutes and filtered through 0.22 um Steriflip filter (Millipore Sigma). Samples were then ultracentrifuged at 120,000×g at 4C for 70 minutes, washed with PBS and ultracentrifuged again. The pellet was then resuspended in PBS.

EV Isolation from Plasma or CSF by MMR Slurry

Human plasma (collected in K2-EDTA tubes) or CSF was ordered from BioIVT. All biofluids were spun down at 2000×g for 10 minutes and pellet was discarded. Mixed mode chromatography resin slurry was prepared by taking Capto Core 700 resin (Cytiva) and centrifuging the resin at 800×g for 5 minutes, washing 3 times with PBS in a 50 mL falcon tube, and resuspending in a volume equal to the resin volume of PBS to produce a 50% slurry. For CSF EV isolation, samples were centrifuged at 2000×g for 10 minutes to remove any potential residual cells. The protein concentration of the CSF was then determined using Qubit Protein Assay kit (ThermoFisher Scientific) and a volume of MMR slurry corresponding to the protein content of the samples added. The ratio of MMR slurry was varied, as specified. For mass spectrometry experiments, the ratio of MMR slurry used was 1uL slurry/0.3 ug protein.

The samples were mixed end over end for 45 minutes at room temperature, and then centrifuged at 800×g for 10 min. Finally, the supernatant is transferred and centrifuged in Corning CoStar X 0.45 μm filters at 2000×g for 10 minutes to separate the CSF from the Capto Core beads.

### Total Protein Staining

Protein samples were denatured in LDS (ThermoFisher Scientific) for 10 minutes at 70C before loading on a polyacrylamide protein gel for total protein staining or Western blotting. Bolt Bis-Tris Plus 4 to 12% gels were used and samples were run at 150V for 60 minutes. Coomassie Blue total protein staining was performed on gels using Acqua Stain (Bulldog Bio). The gel was incubated in the stain overnight, washed in deionized water, and then imaged with a Gel Doc EZ Imager (BioRad).

### Western Blotting

Western blotting of EVs was previously described in detail (46). The iBlot2 Dry Blotting System (ThermoFisher Scientific) was used for transfer at 20V for three to seven minutes, depending on the size of the protein marker. The following primary antibodies were used for western blot at the corresponding dilutions: M38 for CD81 (Thermo Fisher Scientific) at 1:666, H5C6 for CD63 (BD) at 1:1000, CD9 (Millipore) at 1:1000, EPR18998 for L1CAM (Abcam) at 1:500, ab47441 for GJA1 at 1:500, F-10 for Albumin (Santa Cruz) at 1:1000, 41 for SYT1 (BD) at 1:500. The blots were incubated on a shaker in milk (5% weight by volume) dissolved in PBS-T solution (PBS with 0.1% Tween) containing the primary antibodies overnight at 4C. The next day, blots were washed thrice with PBS-T, incubated with TrueBlot HRP (Rockland) or cross-adsorbed HRP (Bethyl) secondary antibody at a concentration of 1:2000 in milk buffer for 2 hours, and then washed thrice again. Blots were developed with WesternBright ECL-spray (Advansta) and imaged on a Sapphire Biomolecular Imager (Azure Biosystems).

EV Immuno-Isolation from Cell Culture EVs

Isolation Buffer was prepared by adding BSA to 7.4 pH PBS to final concentration of 1 mg/mL and filtered through a 0.22 μm Steriflip Filter (Millipore). 500 μL (2×10\hat{ }8) Dynabeads Goat Anti-Mouse IgG beads (Thermo Fisher Scientific) were put into 2 mL and placed on a magnetic rack. Supernatant was removed and replaced with 250 μL of Isolation Buffer off of the magnet. 10 μg primary antibody was coupled to beads overnight at 4C with end over end rotation. The following antibodies were used for immuno-isolation: 5G3 (BD) for L1CAM, 1C51 for mCherry (Abcam), 9F9.F9 for GFP (Abcam), 1.3.3.22 (ThermoFisher Scientific) for CD81, and H5C6 (BD) for CD63. On the next day, beads were washed twice with 1 mL of Isolation Buffer each time. EVs were then added (usually one pellet was in 150 μL) and Isolation Buffer was added to bring the volume to 0.5 mL. Immuno-isolation was performed on rotating rack either at 4C for 24 hours for L1CAM or for 1 hour at 37C for CD81 or CD63.

EV Immuno-Isolation from Human Biofluids

EV immuno-isolation from CSF or plasma was performed similarly to EV immuno-isolation from cell culture EVs, with a few minor modifications. PBS pH 7.4 was used without addition of BSA as the isolation buffer. 250 μL (1×10\hat{ }8) Dynabeads Goat Anti-Mouse IgG beads (Thermo Fisher Scientific) or 50 μL (1.5 mg) of Dynabeads Protein A (Thermo Fisher Scientific) were put into a 2 mL tube and placed a magnetic rack. Supernatant was removed, beads are washed with 1 mL isolation buffer, and then brought up to final incubation volume of 0.5 mL with PBS pH 7.4 and 10 μg primary antibody which is coupled rotating end over end to beads overnight at 4C. The following antibodies were used for immuno-isolation: mouse monoclonal CD81 (clone 1.3.3.22, ThermoFisher Scientific), CD63 (clone H5C6, BD Biosciences), CD9 (clone CBL162, Millipore), GFP (clone 1GFP63, Biolegend or clone 9F9.F9, Rockland) and mCherry (clone EPR20579, Abcam). On the next day, beads were washed twice with 1 mL of Isolation Buffer each time. CSF or plasma was centrifuged at 2000×g for 10 minutes, and supernatant was passed through CoStar Spin-X 0.45 μm filters Corning 2000×g for 10 minutes. The CSF and plasma were then spin filtered in Amicon Ultra 2 mL10K centrifugal filter units (Millipore) for 2 hours to reduce volume, so that 1 mL of CSF or plasma can be incubated with each bead isolation to a final volume of 0.5 mL. Immuno-isolation was performed on a rotating rack either for 1 hour at 4C for CD9, CD63 and CD81.

### Mass Spectrometry

Mass Spectrometry for neuron EVs isolated from cell culture was performed at the Broad Institute Proteomics Platform. EVs were lysed in RIPA buffer (Thermo Fisher Scientific). Samples were then run on an SDS gel and band corresponding in size to Albumin was cut out and discarded. Remaining samples were prepared for TMT labeling and run on Mass Spectrometer. Mass Spectrometry for EVs isolated from CSF or plasma using MMR Slurry was performed at the Harvard Center for Proteomics.

### Examples 2: Detection of NRXN3 in Human CSF and Neuron-Specific Extracellular Vesicles

This Example provides an experimental validation for the neuron-specific EV markers identified above in Example 1. Specifically, NRXN3, one of the markers in Table 1, was detected in EVs derived from conditioned media of human iPS-derived neurons, as well as in the human CSF samples.

EVs were isolated from conditioned media of human iPS-derived neurons as described above. Briefly, iNGN cells (Busskamp et al 2014) were grown in mTeSR1 media on Matrigel-coated plates. Doxycycline (Dox) was diluted in PBS and added to mTeSR1 at a final concentration of 0.5 g/mL to initiate differentiation. On Day 4 after Dox addition, media was switched to DMEM with Glutamax supplemented with B27 Serum-Free Supplement and Penicillin Streptomycin. EVs from neurons were collected on Day 6 or Day 7 after Dox addition. EV isolation from cell culture was performed by differential ultracentrifugation. Cell culture media (240 mL per isolation) was centrifuged at 300×g for 10 minutes and supernatant was centrifuged again at 2000×g for 10 minutes. Supernatant was centrifuged at 16,500×g at 4° C. for 20 minutes and filtered through 0.22 μm Steriflip filter. Samples were then ultracentrifuged at 120,000×g at 4° C. for 70 minutes, washed with PBS and ultracentrifuged again. The pellet was then resuspended in PBS.

Human CSF samples (from Brigham and Women's Hospital) were centrifuged at 2000×g for 10 minutes. Next, the supernatant was centrifuged through a 0.45 μm Corning Costar SPIN-X centrifuge tube filter (Sigma-Aldrich) at 2000×g for 10 minutes to get rid of any remaining cells or cell debris. 1 mL of CSF was loaded onto the size exchange chromatography (SEC) Column.

Briefly, Sepharose CL-6B resins were washed with PBS in a glass bottle. The volume of resin was washed three times with an equal volume of PBS before use. Econo-Pac Chromatography columns were packed with resin and a frit was inserted into the column above the resin. Each column was washed with 10 mL PBS (twice 5 mL at a time) prior to loading of sample. For SEC columns, resin was added until the bed volume (resin without liquid) reached 10 mL. Sample (1 mL CSF or neuron EVs) was loaded once PBS from wash had finished going through the column. Once the sample fully entered the column, 0.5 mL fractions were collected.

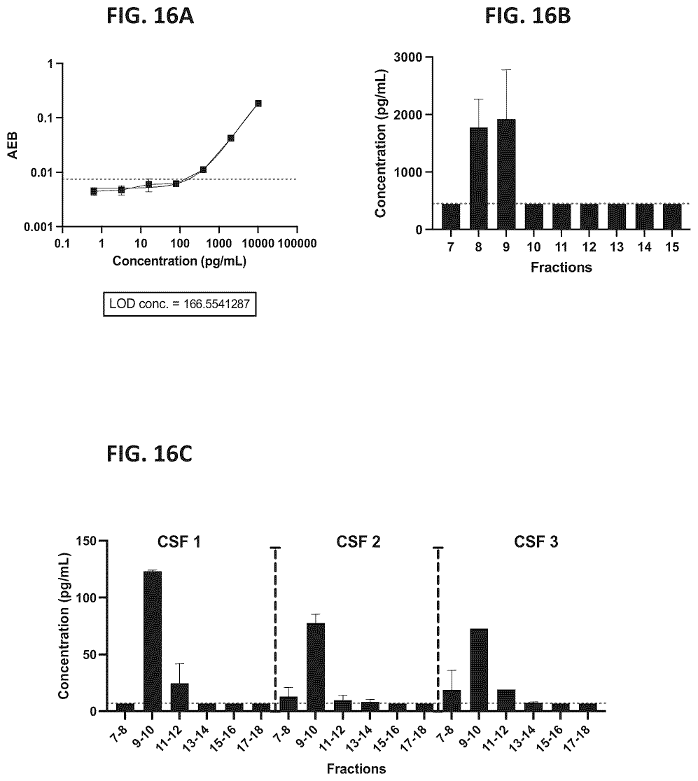

Detection of NRXN3 was performed by Simoa assay (FIG. 16A). Candidate capture antibody (Invitrogen PA5-71367) was coupled to carboxylated paramagnetic beads from the Simoa Homebrew Assay Development Kit (Quanterix) using EDC chemistry (Thermo Fisher Scientific). Candidate detector antibody (CST 480045) was conjugated to biotin using EZ-Link NHS-PEG4 Biotin (Thermo Fisher Scientific). All samples were measured in duplicate using the HD-X analyzer (Quanterix). NRXN3 was measured with a two-step assay. Average Enzyme per Bead (AEB) values were calculated by the HD-X software

As shown in FIG. 16B and FIG. 16C, the NRXN3 marker was detected in early SEC fractions from EVs isolated from conditioned media of human iPS-derived neurons, as well as early SEC fractions in human CSF samples.

These data validate and confirm that markers of the present invention are brain-specific and/or neuron-specific EV markers, and these markers are useful in isolating brain-specific and/or neuron-specific EVs from human samples.

### REFERENCES

1. Shah R, Patel T, Freedman J E. Circulating Extracellular Vesicles in Human Disease. The New England journal of medicine. 2018; 379(10):958-66.
2. Mustapic M, Eitan E, Werner J K, Jr., Berkowitz S T, Lazaropoulos M P, Tran J, et al. Plasma Extracellular Vesicles Enriched for Neuronal Origin: A Potential Window into Brain Pathologic Processes. Front Neurosci. 2017; 11:278.
3. Hornung S, Dutta S, Bitan G. CNS-Derived Blood Exosomes as a Promising Source of Biomarkers: Opportunities and Challenges. Front Mol Neurosci. 2020; 13:38.
4. Abner E L, Jicha G A, Shaw L M, Trojanowski J Q, Goetzl E J. Plasma neuronal exosomal levels of Alzheimer's disease biomarkers in normal aging. Ann Clin Transl Neurol. 2016; 3(5):399-403.
5. Cha D J, Mengel D, Mustapic M, Liu W, Selkoe D J, Kapogiannis D, et al. miR-212 and miR-132 Are Downregulated in Neurally Derived Plasma Exosomes of Alzheimer's Patients. Front Neurosci. 2019; 13:1208.
6. Chawla S, Gulyani S, Allen R P, Earley C J, Li X, Van Zijl P, et al. Extracellular vesicles reveal abnormalities in neuronal iron metabolism in restless legs syndrome. Sleep. 2019; 42(7).
7. Fiandaca M S, Kapogiannis D, Mapstone M, Boxer A, Eitan E, Schwartz J B, et al. Identification of preclinical Alzheimer's disease by a profile of pathogenic proteins in neurally derived blood exosomes: A case-control study. Alzheimer's & dementia: the journal of the Alzheimer's Association. 2015; 11(6):600-7.el.
8. Gill J, Mustapic M, Diaz-Arrastia R, Lange R, Gulyani S, Diehl T, et al. Higher exosomal tau, amyloid-beta 42 and IL-10 are associated with mild TBIs and chronic symptoms in military personnel. Brain Inj. 2018; 32(10):1277-84.
9. Goetzl E J, Abner E L, Jicha G A, Kapogiannis D, Schwartz J B. Declining levels of functionally specialized synaptic proteins in plasma neuronal exosomes with progression of Alzheimer's disease. Faseb j. 2018; 32(2):888-93.
10. Goetzl E J, Boxer A, Schwartz J B, Abner E L, Petersen R C, Miller B L, et al. Low neural exosomal levels of cellular survival factors in Alzheimer's disease. Ann Clin Transl Neurol. 2015; 2(7):769-73.
11. Goetzl E J, Elahi F M, Mustapic M, Kapogiannis D, Pryhoda M, Gilmore A, et al. Altered levels of plasma neuron-derived exosomes and their cargo proteins characterize acute and chronic mild traumatic brain injury. Faseb j. 2019; 33(4):5082-8.
12. Goetzl E J, Kapogiannis D, Schwartz J B, Lobach I V, Goetzl L, Abner E L, et al. Decreased synaptic proteins in neuronal exosomes of frontotemporal dementia and Alzheimer's disease. Faseb j. 2016; 30(12):4141-8.
13. Goetzl E J, Peltz C B, Mustapic M, Kapogiannis D, Yaffe K. Neuron-Derived Plasma Exosome Proteins after Remote Traumatic Brain Injury. J Neurotrauma. 2020; 37(2):382-8.
14. Goetzl L, Darbinian N, Goetzl E J. Novel window on early human neurodevelopment via fetal exosomes in maternal blood. Ann Clin Transl Neurol. 2016; 3 (5):381-5.
15. Guix F X, Corbett G T, Cha D J, Mustapic M, Liu W, Mengel D, et al. Detection of Aggregation-Competent Tau in Neuron-Derived Extracellular Vesicles. International journal of molecular sciences. 2018; 19(3).
16. Hamlett E D, Goetzl E J, Ledreux A, Vasilevko V, Boger H A, LaRosa A, et al. Neuronal exosomes reveal Alzheimer's disease biomarkers in Down syndrome. Alzheimer's & dementia: the journal of the Alzheimer's Association. 2017; 13(5):541-9.
17. Kapogiannis D, Boxer A, Schwartz J B, Abner E L, Biragyn A, Masharani U, et al. Dysfunctionally phosphorylated type 1 insulin receptor substrate in neural-derived blood exosomes of preclinical Alzheimer's disease. Faseb j. 2015; 29(2):589-96.
18. Kapogiannis D, Mustapic M, Shardell M D, Berkowitz S T, Diehl T C, Spangler R D, et al. Association of Extracellular Vesicle Biomarkers With Alzheimer Disease in the Baltimore Longitudinal Study of Aging. JAMA Neurol. 2019; 76(11):1340-51.
19. Nogueras-Ortiz C J, Mahairaki V, Delgado-Peraza F, Das D, Avgerinos K, Eren E, et al. Astrocyte- and Neuron-Derived Extracellular Vesicles from Alzheimer's Disease Patients Effect Complement-Mediated Neurotoxicity. Cells. 2020; 9(7).
20. Pulliam L, Liston M, Sun B, Narvid J. Using neuronal extracellular vesicles and machine learning to predict cognitive deficits in HIV. J Neurovirol. 2020.
21. Pulliam L, Sun B, Mustapic M, Chawla S, Kapogiannis D. Plasma neuronal exosomes serve as biomarkers of cognitive impairment in HIV infection and Alzheimer's disease. J Neurovirol. 2019; 25(5):702-9.
22. Shi M, Kovac A, Korff A, Cook T J, Ginghina C, Bullock K M, et al. CNS tau efflux via exosomes is likely increased in Parkinson's disease but not in Alzheimer's disease. Alzheimer's & dementia: the journal of the Alzheimer's Association. 2016; 12(11):1125-31.
23. Shi M, Liu C, Cook T J, Bullock K M, Zhao Y, Ginghina C, et al. Plasma exosomal α-synuclein is likely CNS-derived and increased in Parkinson's disease. Acta neuropathologica. 2014; 128(5):639-50.
24. Suire C N, Eitan E, Shaffer N C, Tian Q, Studenski S, Mattson M P, et al. Walking speed decline in older adults is associated with elevated pro-BDNF in plasma extracellular vesicles. Exp Gerontol. 2017; 98:209-16.
25. Sun B, Dalvi P, Abadjian L, Tang N, Pulliam L. Blood neuron-derived exosomes as biomarkers of cognitive impairment in HIV. Aids. 2017; 31(14):F9-f17.
26. Winston C N, Goetzl E J, Baker L D, Vitiello M V, Rissman R A. Growth Hormone-Releasing Hormone Modulation of Neuronal Exosome Biomarkers in Mild Cognitive Impairment. J Alzheimers Dis. 2018; 66(3):971-81.
27. Zhao Z H, Chen Z T, Zhou R L, Zhang X, Ye Q Y, Wang Y Z. Increased DJ-1 and α-Synuclein in Plasma Neural-Derived Exosomes as Potential Markers for Parkinson's Disease. Front Aging Neurosci. 2018; 10:438.
28. Ko J, Hemphill M, Yang Z, Beard K, Sewell E, Shallcross J, et al. Multi-Dimensional Mapping of Brain-Derived Extracellular Vesicle MicroRNA Biomarker for Traumatic Brain Injury Diagnostics. J Neurotrauma. 2019.
29. Ko J, Hemphill M, Yang Z, Sewell E, Na Y J, Sandsmark D K, et al. Diagnosis of traumatic brain injury using miRNA signatures in nanomagnetically isolated brain-derived extracellular vesicles. Lab Chip. 2018; 18(23):3617-30.
30. Jiang C, Hopfner F, Katsikoudi A, Hein R, Catli C, Evetts S, et al. Serum neuronal exosomes predict and differentiate Parkinson's disease from atypical parkinsonism. J Neurol Neurosurg Psychiatry. 2020; 91(7):720-9.
31. Zhang N, Gu D, Meng M, Gordon M L. TDP-43 Is Elevated in Plasma Neuronal-Derived Exosomes of Patients With Alzheimer's Disease. Front Aging Neurosci. 2020; 12:166.
32. Norman M, Ter-Ovanesyan D, Trieu W, Lazarovits R, Kowal E J K, Lee J H, et al. L1CAM is not associated with extracellular vesicles in human cerebrospinal fluid or plasma. Nature methods. 2021; 18(6):631-4.
33. Consortium T U. UniProt: the universal protein knowledgebase in 2021. Nucleic Acids Research. 2020; 49(D1) D480-D9.
34. Zhang Y, Sloan S A, Clarke L E, Caneda C, Plaza C A, Blumenthal P D, et al.

Purification and Characterization of Progenitor and Mature Human Astrocytes Reveals Transcriptional and Functional Differences with Mouse. Neuron. 2016; 89(1) 37-53.
35. The GTEx Consortium atlas of genetic regulatory effects across human tissues. Science (New York, N Y). 2020; 369(6509) 1318-30.
36. Yanai I, Benjamin H, Shmoish M, Chalifa-Caspi V, Shklar M, Ophir R, et al. Genome-wide midrange transcription profiles reveal expression level relationships in human tissue specification. Bioinformatics (Oxford, England). 2005; 21(5):650-9.
37. Kryuchkova-Mostacci N, Robinson-Rechavi M. A benchmark of gene expression tissue-specificity metrics. Briefings in bioinformatics. 2017; 18(2):205-14.
38. Busskamp V, Lewis N E, Guye P, Ng A H, Shipman S L, Byrne S M, et al. Rapid neurogenesis through transcriptional activation in human stem cells. Mol Syst Biol. 2014; 10:760.
39. Kowal E J K, Ter-Ovanesyan D, Regev A, Church G M. Extracellular Vesicle Isolation and Analysis by Western Blotting. Methods Mol Biol. 2017; 1660:143-52.
40. Smolarz M, Pietrowska M, Matysiak N, Mielańczyk Ł, Widłak P. Proteome Profiling of Exosomes Purified from a Small Amount of Human Serum: The Problem of Co-Purified Serum Components. Proteomes. 2019; 7(2).
41. Ter-Ovanesyan D, Norman M, Lazarovits R, Trieu W, Lee J H, Church G, et al. Framework for rapid comparison of extracellular vesicle isolation methods. eLife. 2021; 10.
42. Blom H, Åkerblom A, Kon T, Shaker S, van der Pol L, Lundgren M. Efficient chromatographic reduction of ovalbumin for egg-based influenza virus purification. Vaccine. 2014; 32(30):3721-4.
43. James K T, Cooney B, Agopsowicz K, Trevors M A, Mohamed A, Stoltz D, et al. Novel High-throughput Approach for Purification of Infectious Virions. Scientific reports. 2016; 6(1):36826.
44. Van Deun J, Jo A, Li H, Lin H Y, Weissleder R, Im H, et al. Integrated Dual-Mode Chromatography to Enrich Extracellular Vesicles from Plasma. Advanced biosystems. 2020: e1900310.
45. Schutzer S E, Liu T, Natelson B H, Angel T E, Schepmoes A A, Purvine S O, et al. Establishing the proteome of normal human cerebrospinal fluid. PloS one. 2010; 5(6):e10980.
46. Guldbrandsen A, Vethe H, Farag Y, Oveland E, Garberg H, Berle M, et al. In-depth characterization of the cerebrospinal fluid (CSF) proteome displayed through the CSF proteome resource (CSF-P R). Mol Cell Proteomics. 2014; 13(11):3152-63.
47. Deutsch E W, Omenn G S, Sun Z, Maes M, Pernemalm M, Palaniappan K K, et al. Advances and Utility of the Human Plasma Proteome. Journal of proteome research. 2021; 20 (12):5241-63.
48. Muraoka S, Lin W, Chen M, Hersh S W, Emili A, Xia W, et al. Assessment of separation methods for extracellular vesicles from human and mouse brain tissues and human cerebrospinal fluids. Methods. 2020; 177:35-49.
49. Thompson A G, Gray E, Mäger I, Fischer R, Thézénas M L, Charles P D, et al. UFLC-Derived CSF Extracellular Vesicle Origin and Proteome. Proteomics. 2018; 18(24):e1800257.
50. Thompson A G, Gray E, Mäger I, Thézenas M L, Charles P D, Talbot K, et al. CSF extracellular vesicle proteomics demonstrates altered protein homeostasis in amyotrophic lateral sclerosis. Clin Proteomics. 2020; 17:31.
51. Chiasserini D, van Weering J R, Piersma S R, Pham T V, Malekzadeh A, Teunissen C E, et al. Proteomic analysis of cerebrospinal fluid extracellular vesicles: a comprehensive dataset. Journal of proteomics. 2014; 106:191-204.
52. Welsh J A, Killingsworth B, Kepley J, Traynor T, Mckinnon K, Savage J, et al. A simple, high-throughput method of protein and label removal from extracellular vesicle samples. Nanoscale. 2021; 13(6):3737-45.
53. Regev A, Teichmann S A, Lander E S, Amit I, Benoist C, Birney E, et al. The Human Cell Atlas. eLife. 2017; 6.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

The invention claimed is:

1. A method for isolating cell type-specific and/or organ-specific extracellular vesicles from a subject, comprising
(a) obtaining a biological sample from the subject; and
(b) isolating the cell type-specific and/or organ-specific extracellular vesicles based on the presence of a biomarker on the surface of the extracellular vesicles, wherein the biomarker comprises one or more biomarkers selected from Tables 1-5.

2. The method of claim 1, wherein the biological sample comprises a liquid biological sample, optionally selected from the group consisting of whole blood, serum, plasma, cerebrospinal fluid, spinal fluid, amniotic fluid, aqueous humor, vitreous humor, bile, breast milk, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof.

3. (canceled)

4. The method of claim 1,
(a) wherein the extracellular vesicles are brain-specific;
(b) wherein the extracellular vesicles are neuron-specific, astrocyte-specific, oligodendrocyte-specific, and/or microglial-specific;
(c) wherein the extracellular vesicles are neuron-specific and, optionally
wherein the one or more biomarkers are selected from Tables 1 and 5, or
wherein the one or more biomarkers are selected from the group consisting of GABRG2, SVOP, SLC32A1, GRM7, GABRB3, CHRNB2, SLC12A5, GRM8, PTPRT, SLC6A17, PCDHAC2, PLPPR4, C11orf87, SORCS3, CALY, PTPRR, KIAA1549L, HCN1, CDH18, TMEM132D, GPR158, FRRSIL, ATP2B3, GRIA4, ST8SIA3, HS6ST3, SEZ6, and NRXN3;
(d) wherein the extracellular vesicles are astrocyte-specific and, optionally, wherein the one or more biomarkers are selected from Table 2;
(e) wherein the extracellular vesicles are oligodendrocyte-specific and, optionally, wherein the one or more biomarkers are selected from Table 3; and/or
(f) wherein the extracellular vesicles are microglial-specific and, optionally, wherein the one or more biomarkers are selected from Table 4.

5-10. (canceled)

11. The method of claim 1,
(a) wherein the cell type-specific and/or organ-specific EVs are isolated by immuno-isolation, mixed-mode chromatography, size exclusion chromatography, cation exchange chromatography, anion exchange chromatography, gel permeation chromatography, differential centrifugation, sucrose density gradients, organelle electrophoresis, magnetic activated cell sorting (MACS), or nanomembrane ultrafiltration concentrator; and/or
(b) wherein the immuno-isolation comprises a microfluidic affinity-based isolation, a magnetic based isolation, a pull-down isolation, or a fluorescence activated sorting-based isolation.

12. (canceled)

13. A method for isolating brain-specific extracellular vesicles from a subject, comprising
(a) obtaining a biological sample from the subject;
(b) isolating extracellular vesicles from the sample based on the presence of a biomarker on the surface of the extracellular vesicles, wherein the biomarker comprises one or more biomarkers selected from Tables 1-5.

14. The method of claim 13, wherein the biological sample comprises a liquid biological sample, optionally selected from the group consisting of whole blood, serum, plasma, cerebrospinal fluid, spinal fluid, amniotic fluid, aqueous humor, vitreous humor, bile, breast milk, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof.

15. (canceled)

16. The method of claim 13,
(a) wherein the extracellular vesicles are neuron-specific, astrocyte-specific, oligodendrocyte-specific, and/or microglial-specific;
(b) wherein the extracellular vesicles are neuron-specific and, optionally,
wherein the one or more biomarkers are selected from Tables 1 and 5, or
wherein the one or more biomarkers are selected from the group consisting of GABRG2, SVOP, SLC32A1, GRM7, GABRB3, CHRNB2, SLC12A5, GRM8, PTPRT, SLC6A17, PCDHAC2, PLPPR4, C11orf87, SORCS3, CALY, PTPRR, KIAA1549L, HCN1, CDH18, TMEM132D, GPR158, FRRS1L, ATP2B3, GRIA4, ST8SIA3, HS6ST3, SEZ6, and NRXN3;
(c) wherein the extracellular vesicles are astrocyte-specific and, optionally, wherein the one or more biomarkers are selected from Table 2;
(d) wherein the extracellular vesicles are oligodendrocyte-specific and, optionally, wherein the one or more biomarkers are selected from Table 3; and/or
(e) wherein the extracellular vesicles are microglial-specific and, optionally, wherein the one or more biomarkers are selected from Table 4.

17-21. (canceled)

22. The method of claim 13,
(a) wherein the brain-specific EVs are isolated by immuno-isolation, mixed-mode chromatography, size exclusion chromatography, cation exchange chromatography, anion exchange chromatography, gel permeation chromatography, differential centrifugation, sucrose density gradients, organelle electrophoresis, magnetic activated cell sorting (MACS), or nanomembrane ultrafiltration concentrator; and/or
(b) wherein the immuno-isolation comprises a microfluidic affinity-based isolation, a magnetic based isolation, a pull-down isolation, or a fluorescence activated sorting-based isolation.

23. (canceled)

24. A method for identifying an extracellular vesicle derived from a brain cell, comprising
(a) obtaining a biological sample comprising the extracellular vesicle;
(b) determining the presence or absence of a biomarker on the surface of the extracellular vesicle, wherein the biomarker comprises one or more biomarkers selected from Tables 1-5; and wherein the presence of the biomarker is indicative of the extracellular vesicle being derived from a brain cell.

25. (canceled)

26. The method of claim 24,
wherein the brain cell is a neuron,
optionally, wherein the biomarker comprises one or more biomarkers selected from Tables 1 and 5 or wherein the one or more biomarkers are selected from the group consisting of GABRG2, SVOP, SLC32A1, GRM7, GABRB3, CHRNB2, SLC12A5, GRM8, PTPRT, SLC6A17, PCDHAC2, PLPPR4, C11orf87, SORCS3, CALY, PTPRR, KIAA1549L, HCN1, CDH18, TMEM132D, GPR158, FRRS1L, ATP2B3, GRIA4, ST8SIA3, HS6ST3, SEZ6, and NRXN3; and wherein the presence of the biomarker is indicative of the extracellular vesicle being derived from a neuron.

27. (canceled)

28. The method of claim 24, wherein the brain cell is an astrocyte, optionally, wherein the biomarker comprises one or more biomarkers selected from Table 2; and wherein the presence of the biomarker is indicative of the extracellular vesicle being derived from an astrocyte.

29. The method of claim 24, wherein the brain cell is an oligodendrocyte,
wherein the biomarker comprises one or more biomarkers selected from Table 3; and wherein the presence of the biomarker is indicative of the extracellular vesicle being derived from an oligodendrocyte.

30. The method of claim 24, wherein the brain cell is a microglial cell,
optionally, wherein the biomarker comprises one or more biomarkers selected from Table 4; and wherein the presence of the biomarker is indicative of the extracellular vesicle being derived from a microglial cell.

31. The method of claim 24,
(a) wherein the biological sample comprises a liquid biological sample, optionally selected from the group consisting of whole blood, serum, plasma, cerebrospinal fluid, spinal fluid, amniotic fluid, aqueous humor, vitreous humor, bile, breast milk, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof; and/or
(b) wherein the biological sample is obtained from a subject; and/or
(c) wherein the presence or absence of the biomarker is determined by RNA sequencing (RNA seq), DNA sequencing, array analysis, reverse transcription polymerase chain reaction (RT-PCR), quantitative reverse transcription polymerase chain reaction (qRT-PCR), proteomic profiling, mass spectrometry, immunoassay, ELISA, fluorescence activated cell sorting (FACS), SDS-polyacrylamide gel electrophoresis (SDS-PAGE), or Western blot analysis.

32-34. (canceled)

35. A method for diagnosing, prognosing, or identifying a subject at risk of developing a neurodegenerative disorder in a subject comprising:
(a) obtaining a biological sample from the subject;
(b) isolating brain-specific extracellular vesicles from the biological sample based on the presence of a biomarker in the isolated extracellular vesicles, wherein the biomarker comprises one or more biomarkers selected from Tables 1-5;
(c) extracting protein and/or nucleic acids from the isolated brain-specific extracellular vesicles; and
(d) analyzing the extracted protein and/or nucleic acids from the isolated brain-specific extracellular vesicles, thereby diagnosing, prognosing, or identifying the subject at risk of developing the neurodegenerative disorder.

36. The method of claim 35, wherein the biological sample comprises a liquid biological sample, optionally selected from the group consisting of whole blood, serum, plasma, cerebrospinal fluid, spinal fluid, amniotic fluid, aqueous humor, vitreous humor, bile, breast milk, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof.

37. (canceled)

38. (canceled)

39. The method of claim 35,
(a) wherein the extracellular vesicles are neuron-specific and, optionally, wherein the one or more biomarkers are selected from Tables 1 and 5, or
wherein the one or more biomarkers are selected from the group consisting of GABRG2, SVOP, SLC32A1, GRM7, GABRB3, CHRNB2, SLC12A5, GRM8, PTPRT, SLC6A17, PCDHAC2, PLPPR4, C11orf87, SORCS3, CALY, PTPRR, KIAA1549L, HCN1, CDH18, TMEM132D, GPR158, FRRS1L, ATP2B3, GRIA4, ST8SIA3, HS6ST3, SEZ6, and NRXN3;
(b) wherein the extracellular vesicles are astrocyte-specific and, optionally, wherein the one or more biomarkers are selected from Table 2;
(c) wherein the extracellular vesicles are oligodendrocyte-specific and, optionally, wherein the one or more biomarkers are selected from Table 3; and/or
(d) wherein the extracellular vesicles are microglial-specific and, optionally, wherein the one or more biomarkers are selected from Table 4.

40-43. (canceled)

44. The method of claim 35,
(a) wherein the extracted nucleic acids comprise messenger RNA (mRNA), microRNA (miRNA), long non-coding RNA (lncRNA), small non-coding RNA, DNA, and any other full length or fragment of RNAs or DNAs;
(b) wherein analyzing the extracted nucleic acids from the isolated brain-specific extracellular vesicles comprises RNA sequencing (RNA seq), DNA sequencing, array analysis, reverse transcription polymerase chain reaction (RT-PCR), or quantitative reverse transcription polymerase chain reaction (qRT-PCR);
(c) wherein analyzing the extracted nucleic acids from the isolated brain-specific extracellular vesicles comprises genome-wide analysis, or transcriptome profiling;
(d) wherein analyzing the extracted nucleic acids from the isolated brain-specific extracellular vesicles comprises analyzing a gene of interest, wherein the gene of interest is associated with the neurodegenerative disorder, and/or testing for the presence or absence of said gene of interest, analyzing for one or more allelic variants or mutations of the gene of interest, testing for presence or absence of the allelic variants or mutations;
(e) wherein analyzing the extracted protein from the isolated brain-specific extracellular vesicles comprises proteomic profiling, mass spectrometry, immunoassay, ELISA, fluorescence activated cell sorting (FACS), SDS-polyacrylamide gel electrophoresis (SDS-PAGE), or Western blot analysis;
(f) wherein analyzing the extracted protein from the isolated brain-specific extracellular vesicles comprises analyzing a protein of interest, wherein the protein of interest is associated with the neurodegenerative disorder; and/or testing for the presence or absence of said protein of interest, analyzing for one or more mutations in the protein of interest, testing for presence or absence of the mutations; and/or
(g) wherein the neurodegenerative disorder is selected from the group consisting of: Alzheimer's disease (AD), Huntington's Disease, multiple sclerosisvascular disease dementia, frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease.

45-57. (canceled)
